SEQUENCE LISTING (19 sequences)

SEQ ID NO: 1 - DNA, 22 bp, Artificial sequnece
  Synthetic shRNA polynucleotide
cggctgtgattgacttgaataa

SEQ ID NO: 2 - DNA, 22 bp, Artificial sequence
  Synthetic shRNA polynucleotide
acaccgagaaagacaaagtcaa

SEQ ID NO: 3 - DNA, 22 bp, Artificial sequence
  Synthetic shRNA polynucleotide
ccaaagtcaactgttcatttta

SEQ ID NO: 4 - DNA, 22 bp, Artificial sequence
  Synthetic shRNA polynucleotide
actagaaagtgttgtagttgat

SEQ ID NO: 5 - DNA, 22 bp, Artificial sequence
  Synthetic shRNA polynucleotide
cctgctagaaagtgttgtagtt

SEQ ID NO: 6 - DNA, 22 bp, Artificial sequence
  Synthetic shRNA polynucleotide
acctcttgaacatttaccgtaa

SEQ ID NO: 7 - DNA, 22 bp, Artificial sequence
  Synthetic shRNA polynucleotide
actagaaagtgttgtagttgat

SEQ ID NO: 8 - DNA, 22 bp, Artificial sequence
  Synthetic shRNA polynucleotide
acaccgagaaagacaaagtcaa

SEQ ID NO: 9 - DNA, 22 bp, Artificial sequence
  Synthetic shRNA polynucleotide
cgcccactagtctcatattatt

SEQ ID NO: 10 - DNA, 22 bp, Artificial sequence
  Synthetic shRNA polynucleotide
agcatttacttgtttaacactt

SEQ ID NO: 11 - DNA, 22 bp, Artificial sequence
  Synthetic shRNA polynucleotide
actcaaccaagatcttctaaaa

SEQ ID NO: 12 - DNA, 22 bp, Artificial sequence
  Synthetic shRNA polynucleotide
actctaagacagacctcttcta

SEQ ID NO: 13 - DNA, 22 bp, Artificial sequence
  Synthetic shRNA polynucleotide
atggcaagtgtgtgatttgtga

SEQ ID NO: 14 - DNA, 22 bp, Artificial sequence
  Synthetic shRNA polynucleotide
atcggaagactgtgtgaaaaat

SEQ ID NO: 15 - DNA, 22 bp, Artificial sequence
  Synthetic shRNA polynucleotide
ataagacagacctcttctatga

SEQ ID NO: 16 - DNA, 22 bp, Artificial sequence
  Synthetic shRNA polynucleotide
ccggagaaaacttgatagatta

SEQ ID NO: 17 - DNA, 22 bp, Artificial sequecne
  Synthetic shRNA polynucleotide
cccaggagtgcctgctagtgta

SEQ ID NO: 18 - DNA, 22 bp, Artificial sequence
  Synthetic shRNA polynucleotide
acacggtgttccttatgttata

SEQ ID NO: 19 - DNA, 22 bp, Artificial sequence
  Synthetic shRNA polynucleotide
acaagagaggagtgataattaa

(19) United States
(12) Patent Application Publication
(10) Pub. No.: US 2015/0025017 A1
(43) Pub. Date: Jan. 22, 2015

(54) COMPOSITIONS AND METHODS FOR TREATING CANCER

(71) Applicant: Fred Hutchinson Cancer Research Center, Seattle, WA (US)

(72) Inventors: Christopher G. Hubert, Shoreline, WA (US); Patrick J. Paddison, Seattle, WA (US); James M. Olson, Seattle, WA (US); Robert K. Bradley, Seattle, WA (US)

(21) Appl. No.: 14/381,965

(22) PCT Filed: Feb. 28, 2013
(86) PCT No.: PCT/US13/28423

§ 371 Date: Aug. 28, 2014

Related U.S. Application Data
(60) Provisional application No. 61/604,505, filed on Feb. 28, 2012.
(60) Provisional application No. 61/712,725, filed on Oct. 11, 2012.

(51) Int. Cl. C12N 15/113; G01N 33/574; A61K 45/06

(52) U.S. Cl. CPC C12N 15/113; A61K 45/06; G01N 33/574; C12N 2310/531; G01N 2500/10; G01N 2500/04
USPC 514/19.3; 514/44 A; 435/7.1

(57) ABSTRACT

The present disclosure provides compositions and methods for treating cellular hyperproliferative disorders with a PHF5α inhibitor, such as siRNA, shRNA, antisense oligonucleotides, or pharmaceutical compounds. Exemplary cellular hyperproliferative disorders that can be treated with the PHF5α antagonists of the present disclosure include cancers, such as gliomas, adenocarcinomas, cervical cancer or prostate cancer.

### CROSS-REFERENCE

This application claims the benefit of U.S. Provisional Application No. 61/712,725, filed Oct. 11, 2012, and U.S. Provisional Application No. 61/604,505, filed Feb. 28, 2012, each of which is incorporated herein by reference.

### STATEMENT AS TO FEDERALLY SPONSORED RESEARCH

This invention was made with the support of the United States government under Contract number Grant No. T32 CA080416 by National Institutes of Health.

### SEQUENCE LISTING

The instant application contains a Sequence Listing which has been submitted in ASCII format via EFS-Web and is hereby incorporated by reference in its entirety. Said ASCII copy, created on Feb. 22, 2013, is named 44189-702.601.txt and is 5 Kilobytes in size. No new matter has been added.

### BACKGROUND OF THE INVENTION

The majority of protein-coding genes in the human genome are composed of multiple exons (coding regions) that are separated by introns (non-coding regions). Gene expression results in a single precursor messenger RNA (pre-mRNA). The intron sequences are subsequently removed from the pre-mRNA by a process called splicing, which results in the mature messenger RNA (mRNA). By including different combinations of exons, alternative splicing gives rise to mRNAs encoding distinct protein isoforms. The spliceosome, an intracellular complex of multiple proteins and ribonucleoproteins, catalyzes splicing. Recently, two natural compounds interfering with spliceosome activity were found to display anti-cancer activity (Kaida et al, 2007; Kotake et al, 2007).

Glioma is a type of cancer that originates in the brain or spine. The most invasive and aggressive grade of glioma, glioblastoma multiforme (GBM), is the most common type of brain cancer. GBM is notoriously drug and radiation resistant. Therapies and trials using single agents over the last two decades have repeatedly failed to substantially increase the survival of patients (Asbury, et al., Ed., Diseases of the Nervous System: Clinical Neuroscience and Therapeutic Principals, Vol. 2, Cambridge Univ. Press, Third Edition, pp. 1431-1466). Although new combination therapies may have the potential to improve treatment efficacy by exploiting synergies between drugs, sophisticated strategies to identify glioblastoma therapies have been precluded by several technical and biological barriers (see, e.g., Lee et al., Cancer Cell 9:391, 2006; Li et al., Mol. Cancer Res. 6:21, 2008).

### SUMMARY OF THE INVENTION

In one aspect, the present disclosure relates to compositions and methods for treating cancer and, more particularly, to antagonists of one or more spliceosome proteins PHF5α, U2AF1, or DDX1 to induce cell cycle arrest or inhibit RNA processing in cellular hyperproliferative disorders, such as cancer (e.g., glioma).

In one aspect, the present disclosure provides methods for treating a cellular hyperproliferative disorder associated with an oncogenic pathway (e.g., an aberrant Ras pathway), wherein a subject in need thereof is administered a therapeutically effective amount of a PHF5α antagonist, U2AF1 antagonist, DDX1 antagonist, or a combination thereof. Exemplary PHF5α inhibitors include nucleotide sequences as set forth in any one of SEQ ID NOS.:9, 10, 11, 12, 13, 14, 15, 16, or 17. An exemplary DDX1 inhibitor comprises a nucleotide sequence as set forth in SEQ ID NO.:18. Exemplary U2AF1 inhibitors include nucleotide sequences as set forth in any one of SEQ ID NOS.:1, 2, 3, 4, 5, 6, 7, or 8. Such antagonists may be formulated as compositions and may be combined with other active ingredients, such as chemotherapeutics or antagonists of other target molecules.

In another aspect, the present invention includes a method for treating a cellular hyperproliferative disorder associated with an oncogenic pathway, the method including a) identifying at least one candidate agent that is a PHF5α antagonist, a U2AF1 antagonist, a DDX1 antagonist, or a combination thereof; b) determining whether a subject is suffering from a cellular hyperproliferative disorder associated with an oncogenic pathway (e.g., an aberrant Ras pathway); and c) if the subject is suffering from a cellular hyperproliferative disorder associated with an oncogenic pathway (e.g., an aberrant Ras pathway), administering the subject in need thereof a therapeutically effective amount of the PHF5α antagonist, the U2AF1 antagonist, the DDX1 antagonist, or the combination thereof. In some embodiments, the identifying can include a) providing at least one candidate agent; b) contacting the at least one candidate agent with proliferating cancer cells having an oncogenic pathway (e.g., an aberrant Ras pathway); c) determining whether the at least one candidate agent inhibits proliferation of the cancer cells, wherein inhibition indicates inhibition of PHF5α, U2AF1, DDX1, or a combination thereof, by the at least one candidate agent; and d) determining whether the at least one candidate agent binds to PHF5α, U2AF1, DDX1, or a combination thereof, thereby identifying the PHF5α antagonist, U2AF1 antagonist, DDX1 antagonist, or the combination thereof. In some embodiments, the identifying can include a) providing at least one candidate agent; b) contacting the at least one candidate agent with proliferating cancer cells having an oncogenic pathway (e.g., an aberrant Ras pathway); c) determining whether cell cycle arrest, dysregulated cell cycle progression, dysregulated RNA processing, or a combination thereof is generated in proliferating cancer cells due to inhibition of PHF5α, U2AF1, DDX1, or a combination thereof, wherein cell cycle arrest, dysregulated cell cycle progression, dysregulated RNA processing, or a combination thereof indicates inhibition of PHF5α, U2AF1, DDX1, or a combination thereof by the at least one candidate agent; and d) determining whether the at least one candidate agent binds to PHF5α, U2AF1, DDX1, or a combination thereof, thereby identifying the PHF5α antagonist, U2AF1 antagonist, DDX1 antagonist, or the combination thereof.

In yet another aspect, the present invention includes a method for treating a cellular hyperproliferative disorder associated with an oncogenic pathway, wherein a subject in need thereof is administered a therapeutically effective amount of a PHF5α antagonist, U2AF1 antagonist, DDX1 antagonist, or a combination thereof.

In yet another aspect, the present invention includes a method for identifying a PHF5α antagonist, a U2AF1 antagonist, a DDX1 antagonist, the method including a) providing at least one candidate agent; b) contacting the at least one candidate agent with proliferating cancer cells having an oncogenic pathway (e.g., an aberrant Ras pathway); c) determining whether the at least one candidate agent inhibits proliferation of the cancer cells, wherein inhibition indicates inhibition of PHF5α, U2AF1, DDX1, or a combination thereof, by the at least one candidate agent, thereby identifying the PHF5α antagonist, U2AF1 antagonist, DDX1 antagonist, or the combination thereof. In some embodiments, the method further includes d) determining whether the at least one candidate agent binds to PHF5α, U2AF1, DDX1, or a combination thereof, thereby identifying the PHF5α antagonist, U2AF1 antagonist, DDX1 antagonist, or the combination thereof.

In yet another aspect, the present invention includes a method for identifying a PHF5α antagonist, a U2AF1 antagonist, a DDX1 antagonist, or a combination thereof, the method including a) providing at least one candidate agent; b) contacting the at least one candidate agent with proliferating cancer cells; c) determining whether cell cycle arrest, dysregulated cell cycle progression, dysregulated RNA processing, or a combination thereof is generated in proliferating cancer cells due to inhibition of PHF5α, U2AF1, DDX1, or a combination thereof, wherein cell cycle arrest, dysregulated cell cycle progression, dysregulated RNA processing, or a combination thereof indicates inhibition of PHF5α a, U2AF1, DDX1, or a combination thereof by the at least one candidate agent, thereby identifying the PHF5α antagonist, U2AF1 antagonist, DDX1 antagonist, or the combination thereof. In some embodiments, the methods include d) determining whether the at least one candidate agent binds to PHF5α, U2AF1, DDX1, or a combination thereof, thereby identifying the PHF5α antagonist, U2AF1 antagonist, DDX1 antagonist, or the combination thereof.

In yet another aspect, the present invention includes a method for identifying a SF3b antagonist, the method including a) providing at least one candidate agent; b) contacting the at least one candidate agent with proliferating cancer cells having an oncogenic pathway (e.g., an aberrant Ras pathway); c) determining whether the at least one candidate agent inhibits proliferation of the cancer cells, wherein inhibition indicates inhibition of SF3b, by the at least one candidate agent, thereby identifying the SF3b antagonist. In some embodiments, the methods include d) determining whether the at least one candidate agent binds to SF3b, thereby identifying the SF3b antagonist.

In yet another aspect, the present invention includes a method for identifying a SF3b antagonist, the method including a) providing at least one candidate agent; b) contacting the at least one candidate agent with proliferating cancer cells; c) determining whether cell cycle arrest, dysregulated cell cycle progression, dysregulated RNA processing, or a combination thereof is generated in proliferating cancer cells due to inhibition of SF3b, wherein cell cycle arrest, dysregulated cell cycle progression, dysregulated RNA processing, or a combination thereof indicates inhibition of SF3b by the at least one candidate agent, thereby identifying the SF3b antagonist. In some embodiments, the methods include d) determining whether the at least one candidate agent binds to SF3b, thereby identifying the SF3b antagonist.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B show that knocking down PHF5α protein levels inhibits human glioma neural stem cell (GSC) proliferation. (A) is a bar graph illustrating a greater reduction in proliferation of GSCs compared to normal neural stem cells (NSCs) when PHF5α levels are reduced by exposure of GSCs or NSCs to shRNAs that inhibit production of PHF5α (shPHF5α-861 (SEQ ID NO.:9), shPHF5α-401 (SEQ ID NO.:10), shPHF5α-133 (SEQ ID NO.:11)). (B) is an immunostained western blot showing PHF5α protein levels are reduced in GSCs (G166, 0827, 0131) and NSCs when exposed to a short hairpin RNA (shRNA) that reduces PHF5α RNA levels (shPHF5α-133 (SEQ ID NO.:11), shPHF5α-402 (SEQ ID NO.:10), shPHF5α-861 (SEQ ID NO.:9)) relative to the levels of PHF5α in cells treated with a non-silencing control shRNA (shCtrl).

FIG. 2 shows a cartoon illustration of PHF5α acting as a bridge between U2AF1 and RNA helicase DDX1 (RNA helicase, DDX1) in the context of a U2 snRNP (left side) along with images of GFP-expressing GSCs (right side) that have undergone cell cycle arrest when treated with inhibitor shU2AF1-100 (top) (SEQ ID NO.:1), shPHF5α-861 inhibitor (middle) (SEQ ID NO.:9), or a DDX1-301 inhibitor (SEQ ID NO.:18). Arrows point to rounded cells, which are those showing a cell arrest phenotype.

FIG. 3A shows the quantification of phospho-MPM2 staining in GSCs and NSCs infected with control or shPHF5A virus. (* denotes p-value<0.001; # denotes insignificant p-value=0.5).

FIG. 3B shows viability of GSC or NSC cells treated with increasing doses of SSA. (* denotes p-value<0.0003).

FIG. 3C shows G166 or CB660 cells that were exposed to a dilution series of SSA, SudC1, or SudE-OH (inactive alcohol form of Sudemycin) for 24 hours and then assayed for cell viability by AlamarBlue assay (Invitrogen) at 72 hours after exposure.

FIG. 4A-4D show that targeted knockdown of PHF5α affects both the CD15+ subpopulation of GSCs in two different primary cultures (G166 and 0827). (A) and (C) show the shCtrl (control shRNA) treated cells, while (B) and (D) show the shPHF5α treated cells.

FIGS. 5A-5E show how GSCs were prepared and used for an orthotopic brain xenograft in mice. (A) shows how GSC containing shPHF5α and control shChFP were prepared and used to treat GSCs prior to propagation in culture or in brain xenografts. (B) and (D) show that both the shChFP (red) and shPHF5α (green) treated cells were alive and attached to the culture plate surface two days after culturing. (C) and (E) show the shChFP and shPHF5α treated cells after 12 days in culture.

FIG. 6 shows that primary GBM cells depleted for PHF5α cannot efficiently contribute to tumor formation in vivo. Primary cultures of glioblastoma cells (“0131”) infected with GFP expressing shRNA virus against PHF5α (shPHF5α) or non-silencing control (shCtrl) were mixed with 10% mChFP expressing 0131 cells (“tagged” to express a red fluorescent protein) before orthotopic injection into the right cortex of mouse brain. Tumors grew in all mice injected with 0131 cells (0131 shCtrl, also a chlorotoxin:Cy5.5 fluorescent conjugate that binds to all GBM cells was visualized by a Xenogen® IVIS® system; data not shown), however GFP-expressing shPHF5α cells were unable to contribute to formation of orthotopic tumors (0131 shPHF5α) and yielded tumor masses dominated by mChFP control cells (>90%) with little to no detectible GFP expression when the same brains were visualized for mChFP fluorescence (data not shown).

FIG. 7 shows that inducible shPHF5A inhibition significantly extends the survival of mice having a glioblastoma brain xenograft.

FIG. 8A-8B show the flank xenograft volume over time of GSC-0131 clones expressing doxycycline-inducible PHF5A shRNA or Ctrl shRNA. Tumors were allowed to progress in absence of Dox until the tumor volume of each cohort averaged approximately 75 mm³. Mice were then randomized onto continuous doxycycline or vehicle treatment and tumor volume was monitored over time.

FIG. 8C shows the Kaplan-Meier analysis of mice bearing brain xenografts of doxycycline-inducible PHF5A KD GSCs. At the first sign of symptoms in the first mouse (Day 52; CTX:Cy5.5 image, inset) mice were randomized onto continuous doxycycline or vehicle treatment and survival was monitored over time. Photographs of representative mice from each cohort are shown.

FIG. 9A shows the normalized cell viability of IMR90 fibroblast cells (IMR90) and IMR90 cells infected with a retrovirus encoding the E1A and Ras oncogenes following treatment with sudemycin C1 (an inhibitor of SAP155, which is a component of the U2 snRNP). Normal IMR90 cells without Ras (IMR90) are relatively resistant to both PHF5α knockdown (not shown) and to Sudemycin treatment, whereas the same cells with the addition of oncogenic Ras signaling were greatly sensitized to the same treatments (IMR90+E1A/Ras), demonstrating that a requirement for PHF5α expression and SF3B/U2 snRP function may be generalizable to many tumors driven by oncogenic Ras/PI3-kinase pathway signaling.

FIG. 9B depicts the cellular viability of IMR90 fibroblasts with or without expression of RasV12 after knockdown of PHF5A.

FIG. 9C shows the viability of IMR90 cells with or without RasV12 expression after different exposure time to SudC1.

FIG. 10A-B shows the cellular viability of normal human astrocytes (NHA) or mouse fibroblasts (FIG. 10C) with or without expression of the RasV 12 oncogene after treatment with increasing doses of SudC1 (10A and 10C) or SSA (10B).

FIG. 11 shows that cells expressing Ras were differentially sensitive to Spliceostatin A, Pladienolide B, and Sudemycin C1, all of which target the SF3b complex (7 protein members including PHF5A). The cells expressing Ras were not differentially sensitive to Clotrimazole, chlorhexidine, TG003 and flunarizine.

FIG. 12 shows that expression of activated MEK (downstream of Ras in the signaling cascade) partially duplicates the splicing inhibitor sensitivity seen in cells expressing oncogenic Ras.

FIG. 13 shows a cartoon of a few common patterns of alternative splicing of pre-mRNA (top panel) and shows the results of cDNA sequencing of mRNA (RNA-seq) isolated from NSCs (CB660 NSC) or glioma (G166 Glioma) cells treated with shCtrl or shPHF5α (bottom panels). GSC and NSC primary cultures were infected with shCtrl or shPHF5α virus and were selected with puromycin. RNA was isolated and cDNA library sequencing was performed using an Illumina® Genome Analyzer IIx. The “volcano” plots illustrate relative isoform ratios of (shPHF5α knockdown/shCtrl)×100 for skipped or cassette exon alternative splicing events, wherein shifts to the left indicate increased exon skipping following PHF5α knockdown. The threshold of significance is shown by the dotted line (i.e., dots appearing above the dotted line are statistically significant, wherein each dot corresponds to a particular splicing event). Statistical analysis of the relative abundance of RNA isoforms in shPHF5α knockdown cells as compared to shCtrl cells demonstrated that there was a global increase in exon skipping following shPHF5α in cancer cells, but not benign cells. These results indicate that GSCs, but not normal NSCs, are particularly sensitive to perturbation of the 3′-splice site machinery, resulting in dysregulated RNA processing.

FIGS. 14A-14L show that knockdown of PHF5α results in global splicing defects detectable in GSCs and not in NSCs. (A) Select genes important for cell cycle progression, such as CDC20, display broad splicing defects following PHF5α knockdown. Plot illustrates the density of RNA-seq reads crossing splice junctions and was created with IGV (Robinson et al., Nat. Biotechnol. 29:24, 2011). Aberrant isoforms lacking constitutive exons appear following knockdown of PHF5α with two distinct shRNAs. (B-C) Knockdown of PHF5α results in few changes in usage of competing 5′-splice sites, (D-E) competing 3′-splice sites, or (F-G) mutually exclusive exons in either NSCs or GSCs. (H-I) However, PHF5α knockdown causes a broad increase in skipping of cassette exons in GSCs, but not NSCs. (J) Many of the splicing changes induced by PHF5α knockdown in GSCs introduce in-frame stop codons. Gene expression values were computed with RSEM (Li and Dewey, BMC Bioinformatics 12:323, 2011) and normalized with the TMM method (Robinson and Oshlack, Genome Biol. 11:R25, 2010). Confidence intervals indicate the 1^{st} and 3^{rd} quartiles of expression. (K) PHF5α knockdown causes a dramatic increase in mis-splicing of constitutive junctions, (L) as well as retention of constitutive introns.

FIGS. 15A-15C show that PHF5α is required for proper recognition of an unusual class of exons. (A) Constitutive junctions that are mis-spliced following PHF5α knockdown in GSCs (center) have slightly shorter polypyrimidine tracts than do unaffected constitutive junctions (top); in contrast, retained constitutive introns (bottom) have unusually C-rich polypyrimidine tracts. (B) Retained constitutive introns tend to be much shorter. Plot illustrates the median intron length, and error bars indicate the standard error estimated by bootstrapping. (C) Retained constitutive introns have branch points that are unusually proximal to the 3′-splice site. Box plots indicate 1^{st} and 3^{rd}quartiles of the first upstream AG, a proxy for the branch point location (Gooding et al., Genome Biol. 7:R1, 2006).

FIG. 16 shows gels identifying that treatment of GSCs with SudC1 resulted in dose-dependent GSC-specific splicing.

### DETAILED DESCRIPTION OF THE INVENTION

In one aspect, the present invention provides methods for identifying antagonists of PHF5α, U2AF1, DDX1, SF3b, and associated methods of treatment for, e.g., cellular hyperproliferative disorders.

In one aspect, the present disclosure provides methods for treating a cellular hyperproliferative disorder (e.g., cancer) associated with an oncogenic pathway (e.g., an aberrant Ras pathway (also referred to as Ras/PI3K pathway) or aberrant Ras pathway signaling), wherein a subject in need thereof is administered a therapeutically effective amount of a PHF5α antagonist or inhibitor, U2AF1 antagonist or inhibitor, DDX1 antagonist or inhibitor, or a combination thereof. Such antagonists or inhibitors include, for example, siRNA, shRNA, antisense oligonucleotides, or the like. Alternatively, a PHF5α antagonist, U2AF1 antagonist, or DDX1 antagonist may be a pharmaceutical compound, such as morpholino antisense oligonucleotides to inhibit interactions between regulatory sequences in the pre-mRNA and core spliceosomal components and regulatory splicing factors, or otherwise inhibit splicing catalysis or trigger defective splicing. An antagonist may also be a modified snRNA, such as a Ua or a U7 snRNA, which can modulate the splicing of select events, or a bifunctional RNA that contains both targeting and regulatory sequences.

Preferably, a PHF5α antagonist, U2AF1 antagonist, DDX1 antagonist, or a combination thereof promotes cell cycle arrest, dysregulated cell cycle progression, dysregulated RNA processing (e.g., global exon skipping, aberrant constitutive junction splicing, constitutive intron retention), or a combination thereof in cells having a hyperproliferative disorder (e.g., cancers such as glioma, colorectal cancer, adenocarcinoma), but does not have such effects or is minimally active against normal cells. In certain embodiments, a PHF5α antagonist, U2AF1 antagonist, DDX1 antagonist, or a combination thereof of the present disclosure is used in combination with other chemotherapeutics or antagonists of other target molecules (e.g., cell division cycle (CDC) proteins, regulator of chromosome condensation (RCC)). In still further embodiments, a PHF5α antagonist, a U2AF1 antagonist, a DDX1 antagonist, or a combination thereof is used in combination with other spliceosome inhibitors, such as sudemycin, spliceostatin, FR901464 or derivatives thereof (such as those disclosed in U.S. Pat. No. 7,825,267, which compounds are herein incorporated by reference), pladienolide, E7107, herboxidine, and derivatives or analogs of each of these compounds.

In further embodiments, the present disclosure provides compositions of a PHF5α antagonist, a U2AF1 antagonist, a DDX1 antagonist, or a combination thereof, wherein the PHF5α antagonist, a U2AF1 antagonist, a DDX1 antagonist, or combination thereof is in a pharmaceutically acceptable diluent, carrier, or excipient.

In certain embodiments, cancers that can be treated with the antagonists and compositions thereof of this disclosure include gliomas (such as glioblastoma multiforme, anaplastic oligodendroglioma, anaplastic astrocytoma), medulloblastoma, myelodysplastic syndrome, central nervous system cancer, skin cancer, melanoma, lung cancer, non-small cell lung cancer, bladder cancer, kidney cancer, urinary tract cancer, urothelial carcinoma, cervical cancer, ovarian cancer, liver cancer, head and neck squamous cell cancer, oral squamous cell cancer, esophageal cancer, gastric cancer, stomach cancer, upper digestive tract cancer, colon cancer, colorectal cancer, seminoma, prostate cancer, breast cancer, endometrial cancer, pancreatic cancer, pancreatic ductal adenocarcinoma, intraductal papillary mucinous neoplasm carcinoma of the pancreas, thyroid cancer, head/neck squamous cell cancer, hematopoietic cancers, lymphoid cancers, leukemias, solid tumors, sarcomas, adenocarcinomas, or the like.

The compounds, compositions and methods of this disclosure allow a person of ordinary skill in the art to more effectively target certain hyperproliferative disorders (such as gliomas, adenocarcinomas, cervical cancer or any other cancer having an activated, upregulated, stimulated, altered or dysregulated Ras pathway or effector), by reducing dosages in single or combination therapies such that normal cells are minimally affected or are unaffected (i.e., toxicity is minimized).

By way of background, the initial RNA transcripts (pre-mRNA) of most eukaryotic genes are retained in the nucleus until non-coding intron sequences are removed by the spliceosome to produce mature messenger RNA (mRNA). The splicing that occurs can vary, so the synthesis of alternative protein products from the same primary transcript can be affected by tissue-specific or developmental signals. A significant fraction of human genetic diseases, including a number of cancers, are believed to result from deviations in the normal pattern of pre-mRNA splicing. The spliceosome is a multi-megadalton complex of ribonucleoprotein (snRNP) particles, which are each composed of one or more uridine-rich small nuclear RNAs and several proteins. The snRNA components of the spliceosome promote the two transesterification reactions of splicing, among other functions.

Two unique spliceosomes coexist in most eukaryotes: the U2-dependent spliceosome, which catalyzes the removal of U2-type introns, and the less abundant U12-dependent spliceosome, which is present in only a subset of eukaryotes and splices the rare U12-type class of introns. The U2-dependent spliceosome is assembled from the U1, U2, U5, and U4/U6 snRNPs and numerous non-snRNP proteins. The U2 snRNP is recruited with two weakly bound protein subunits, SF3a and SF3b, during the first ATP-dependent step in spliceosome assembly. SF3b is composed of seven conserved proteins, including PHF5α, SF3b155, SF3b145, SF3b130, SF3b49, SF3b14a, and SF3b10 (Will et al., EMBO J. 21:4978, 2002).

PHF5α (also referred to herein as PHF5A) contains a Plant Homeo Domain (PHD)-finger-like domain that is flanked by highly basic amino- and carboxy-termini; therefore, PHF5α belongs to the PHD-finger superfamily but it may also act as a chromatin-associated protein. The PHF5α protein bridges the U2 snRNP with the U2AF1 (a U2AF65-U2AF35 heterodimer) associated with the 3′-end of the intron and RNA helicase DDX1 (Rzymski et al., Cytogenet. Genuine Res. 121:232, 2008). This is an important event, as stable U2 snRNP addition is often a regulated step in alternative pre-mRNA splicing. While SF3a and SF3b components appear to be in close contact with the pre-mRNA, the precise function of the individual proteins remain largely unknown. Furthermore, it is unclear whether SF3a and SF3b function outside of the context of the U2 snRNP particle during the spliceosome cycle.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the terms “about” and “consisting essentially of” mean±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

As used herein, “antagonist” or “inhibitor” refers to a compound or combination of compounds that can reduce, minimize, suppress, block, or eliminate expression or function of a target molecule, such as, for example, PHF5α, U2AF1, DDX1, or SF3b. In some embodiments, the PHF5α antagonists, the U2AF1 antagonists, the DDX1 antagonists, the SF3b antagonists, or the combination thereof can directly inhibit activity and/or expression of PHF5α, U2AF1, DDX1, SF3b or a combination thereof. Preferably, an exemplary PHF5α, U2AF1, DDX1, or SF3b antagonist and analogs or derivatives thereof will affect cells exhibiting a hyperproliferative disorder (e.g., cancer) without affecting or minimally affecting normal cells. Exemplary antagonists or inhibitors include polypeptides, polynucleotides, small molecules, or the like. For example, the levels of expression product or level of RNA or equivalent RNA encoding one or more gene products (e.g., PHF5α, U2AF1, DDX1, or SF3b) is reduced below that observed in the absence of a nucleic acid molecule antagonist of the present disclosure. In certain embodiments, inhibition with a nucleic acid molecule capable of mediating RNA interference (e.g., siRNA, shRNA, miRNA) preferably is below that level observed in the presence of an inactive or attenuated molecule that is unable to mediate an RNAi response.

As used herein, “cellular hyperproliferative disorder” refers to a condition, disease, or pathology involving abnormal or uncontrolled cell division. Representative hyperproliferative disorders include neoplasias (e.g., cancer), hyperplasias (e.g., endometrial hyperplasia, benign prostatic hyperplasia), restenosis, cardiac hypertrophy, immune disorders involving, for example, a dysfunctional proliferation response by the cellular immune system, or inflammation. Exemplary cancers in this regard include those listed herein, such as glioma, adenocarcinomas, and cervical cancer. In some embodiments, the cellular hyperproliferative disorders can be associated with an oncogenic pathway. In one aspect, the cellular hyperproliferative disorders can be associated with a Ras oncogene, a Myc oncogene, or other known oncogenes associated with cellular hyperproliferative disorders. In some embodiments, the cellular hyperproliferative disorders can be associated with an aberrant Ras pathway.

As used herein, the terms “derivative” and “analog” when referring to a PHF5α antagonist, U2AF1 antagonist, DDX1 antagonist, a SF3b antagonist, small molecule, polypeptide, siRNA, shRNA, antisense oligonucleotide, or the like, means any such compound that retains essentially the same (at least 50%, and preferably greater than 70%, 80%, or 90%), similar, or enhanced biological function or activity as the original compound. The biological function or activity of such analogs and derivatives can be determined using standard methods (e.g., cell cycle arrest, RNA splicing alteration, protein synthesis inhibition), such as with the assays described herein or known in the art. For example, an analog or derivative may be a pro-drug that can be activated by cleavage to produce an active compound. Alternatively, an analog or derivative thereof can be identified by the ability to specifically bind to a target compound, such as, for example, PHF5α, U2AF1, DDX1, or SF3b.

As used herein, “Ras pathway” or “Ras/PI3K pathway” refers to the various components involved in a cascade of signaling events that couple cell surface receptor activation to downstream effector pathways to control diverse cellular responses, such as proliferation, differentiation and survival. The Ras proteins (e.g., H-ras, K-ras, M-ras, N-ras, R-ras) are signal switch molecules involved in regulating the cascade of signaling events, and can interact directly or indirectly with a variety of effector molecules (e.g., Raf, PI3K, PLC, Ral-GEF, Rassf, IMP). “Ras pathway” or “Ras pathway signaling” may be aberrant when a Ras pathway component or effector is activated, upregulated, stimulated, altered or dysregulated (e.g., one or more of RTK, Ras, Raf, MAPK, MEK, AKT, PI3K, Myc) and results in, for example, a cellular hyperproliferative disorder (e.g., cancer). A “Ras pathway” may be aberrantly activated, upregulated, stimulated, altered or dysregulated in any number of ways, including by one or more mutations in a Ras protein, one or more mutations that alter Ras gene expression, or by one or more alterations in Ras pathway related components or effectors, such as receptor tyrosine kinase (RTK) (e.g., EGFR, VEGF, PDGF, etc.), Raf, mitogen-activated protein kinase (MAPK), extracellular signal-regulated kinase (ERK), MEK, MKK, AKT, phosphatidylinositol 3-kinase (PI3K), Myc, or the like, or any combination thereof. In some embodiments, proteins associated with the pathway can exhibit an altered activation state as a consequence of an up- or down-regulation in expression, or may be more- or less-active as a consequence of a non-mutational change (e.g., such as a change in phosphorylation of one or more tyrosine, threonine, or serine residues in the protein, such as RTK). In certain embodiments, the pathway can include a constitutively activated receptor tyrosine kinase, where it is activated by constant presence of its ligand (e.g., a growth factor).

As used herein, a “subject” is a human or non-human animal. “Subject” also refers to an organism to which a small molecule, chemical entity, nucleic acid molecule, peptide or polypeptide of this disclosure can be administered to inhibit, for example, PHF5α, U2AF1, DDX1, or SF3b. In one embodiment, a subject is a mammal. In another embodiment, a subject is a human, such as a human having or at risk of having a cancer associated with an aberrant Ras pathway (e.g., glioma).

The term “biological sample” includes a blood sample, biopsy specimen, tissue explant, organ culture, biological fluid (e.g., serum, urine, CSF) or any other tissue or cell or other preparation from a subject or a biological source. A biological sample or source may, for example, be a primary cell culture or culture adapted cell line including genetically engineered cell lines that may contain chromosomally integrated or episomal recombinant nucleic acid molecules, somatic cell hybrid cell lines, immortalized or immortalizable cell lines, differentiated or differentiatable cell lines, transformed cell lines, or the like.

In further embodiments of this disclosure, a subject or biological source may be suspected of having or being at risk for having a disease, disorder or condition, including a malignant disease, disorder or condition (e.g., cancer associated with an aberrant Ras pathway, glioma). In certain embodiments, a subject or biological source may be suspected of having or being at risk for having a hyperproliferative disease (e.g., carcinoma, sarcoma), and in certain other embodiments of this disclosure a subject or biological source may be known to be free of a risk or presence of such disease, disorder, or condition.

“Treatment,” “treating” or “ameliorating” refers to either a therapeutic treatment or prophylactic/preventative treatment. A treatment is therapeutic if at least one symptom of disease (e.g., hyperproliferative disorder such as cancer) in an individual receiving treatment improves or a treatment may delay worsening of a progressive disease in an individual, or prevent onset of additional associated diseases (e.g., metastases from cancer).

A “therapeutically effective amount (or dose)” or “effective amount (or dose)” of a PHF5α, U2AF1, DDX1, or SF3b inhibitor, or combination thereof, refers to that amount of compound sufficient to result in amelioration of one or more symptoms of the disease being treated (e.g., cancer associated with an aberrant Ras pathway, glioma) in a statistically significant manner. When referring to an individual active ingredient, administered alone, a therapeutically effective dose refers to that ingredient alone. When referring to a combination, a therapeutically effective dose refers to combined amounts of the active ingredients that result in the therapeutic effect, whether administered serially or simultaneously (in the same formulation or in separate formulations).

The term “pharmaceutically acceptable” refers to molecular entities and compositions that do not produce allergic or other serious adverse reactions when administered to a subject using routes well known in the art.

A “patient in need” or “subject in need” refers to a patient or subject at risk of, or suffering from, a disease, disorder or condition (e.g., cancer associated with an aberrant Ras pathway, glioma) that is amenable to treatment or amelioration with an inhibitor of PHF5α, U2AF1, DDX1, SF3b or a combination thereof, or a composition thereof, as provided herein.

As used herein, “next generation sequencing” refers to high-throughput sequencing methods that allow the sequencing of thousands or millions of molecules in parallel. Examples of next generation sequencing methods include sequencing by synthesis, sequencing by ligation, sequencing by hybridization, polony sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.”

Effective glioblastoma therapies have been precluded by several technical and biological barriers, but recent developments, however, may allow for development of more effective glioma treatments. For example, genetic tools for large-scale screens of mammalian cells have been developed (Paddison et al., Nature 428:427, 2004; Silva et al., Nat. Genet. 37:1281, 2005). Furthermore, new methods for deriving and maintaining human glioma neural stem cells (hGSCs) and normal neural stem cells (NSCs) in a screening-compatible monolayer format was developed (Pollard et al., Cell Stem Cell 4:568, 2009; Sun et al., Mol. Cell. Neurosci. 38:245, 2008). These hGSCs retain specific development potential and genetic alterations unique to the original patient tumors, which provide access to previously inaccessible tumor-specific molecular networks. Finally, a method for growing orthotopic brain cancer xenografts that retain patient-specific molecular signatures and histological features has also been recently developed (Shu et al., Stem Cells 26:1414, 2008).

In certain embodiments, the present disclosure provides methods for treating a subject having a glioma, wherein the subject is administered a therapeutically effective amount of a PHF5α antagonist, U2AF1 antagonist, DDX1 antagonist, or a combination thereof. In further embodiments, such antagonists may be a siRNA, shRNA, miRNA, antisense oligonucleotide, or the like. Alternatively, a PHF5α antagonist, U2AF1 antagonist, or DDX1 antagonist may be a pharmaceutical compound, such as morpholino oligonucleotides to block U2 snRNP function and possibly prevent the splice-directing snRNP complexes from binding to their targets at borders of target introns. Preferably, the PHF5α antagonist, U2AF1 antagonist, or DDX1 antagonist promotes cell cycle arrest, dysregulated cell cycle progression, dysregulated RNA processing (e.g., global exon skipping, aberrant constitutive junction splicing, constitutive intron retention), or a combination thereof in glioma cells, but does not have such effects or is minimally active against normal cells.

In further embodiments, the present disclosure provides methods for treating a subject having a glioma by administering a therapeutically effective amount of a composition comprising a PHF5α antagonist, a U2AF1 antagonist, a DDX1 antagonist, or a combination thereof formulated with a pharmaceutically acceptable diluent, carrier, or excipient.

In certain embodiments, the present disclosure provides methods for treating a subject having a cellular hyperproliferative disorder associated with an oncogenic pathway (e.g., an aberrant Ras pathway or effector), wherein the subject is administered a therapeutically effective amount of a PHF5α antagonist, U2AF1 antagonist, DDX1 antagonist or a combination thereof, or is administered a composition comprising a PHF5α antagonist, a U2AF1 antagonist, a DDX1 antagonist, or a combination thereof formulated with a pharmaceutically acceptable diluent, carrier, or excipient. In preferred embodiments, an aberrant Ras pathway or effector is activated, upregulated, stimulated or otherwise increased, altered or dysregulated such that a cellular hyperproliferative disorder is triggered.

In certain embodiments, a PHF5α antagonist, a U2AF1 antagonist, a DDX1 antagonist, a SF3b antagonist, or a combination thereof, or a composition thereof of the present disclosure is used in combination with other chemotherapeutics or other targeted antagonists or agonist (e.g., enhancing or correcting expression of a tumor suppressor) of interest. In further embodiments, a PHF5α antagonist, a U2AF1 antagonist, a DDX1 antagonist, a combination thereof, or a composition thereof is used in combination with one or more antagonists or inhibitors of ZNF207, POLR21, TFCP2L1, ARL6IP1, C3orf67, CLSTN1, EIF2S1, INTS4, KPNB1, LSM6, PHLDB1, POLR2E, PSMC5, RRM1, RRM2, SNORA21, TRA2B, TRIP13, and VCP. In still further embodiments, the PHF5α antagonist, a U2AF1 antagonist, a DDX1 antagonist, a combination thereof, or a composition thereof is used in combination with other spliceosome inhibitors, such as sudemycin, spliceostatin, FR901464 or derivatives thereof (such as those disclosed in U.S. Pat. No. 7,825,267, which compounds are herein incorporated by reference), pladienolide, E7107, herboxidine, meayamycin, and derivatives or analogs of each of these compounds, and any of the compounds described by Lagisetti et al., J. Med. Chem. 52:6979, 2009, which compounds are hereby incorporated by reference.

Alternatively, exemplary PHF5α antagonists of the present disclosure may be used in combination with U2AF1 inhibitors. The U2 auxiliary factor comprises a large and a small subunit, and is a non-snRNP protein required for the binding of U2 snRNP to the pre-mRNA branch site. The U2AF1 gene encodes the small subunit, which is important for both constitutive and enhancer-dependent RNA splicing.

In further embodiments, PHF5α antagonists of the present disclosure may be used in combination with RNA helicase inhibitors, such as a DDX1 inhibitor. By way of background, DEAD box proteins, characterized by the conserved motif Asp-Glu-Ala-Asp (DEAD), are putative RNA helicases that are implicated in a number of cellular processes involving alteration of RNA secondary structure, including translation initiation, nuclear and mitochondrial splicing, and ribosome and spliceosome assembly. Based on their distribution patterns, some members of this family are believed to be involved in embryogenesis, spermatogenesis, and cellular growth and division. The DDX1 gene encodes a DEAD box protein of unknown function, but it shows high transcription levels in two retinoblastoma cell lines and in tissues of neuroectodermal origin. In this disclosure, DDX1 is shown to be involved in glioma cell proliferation.

Exemplary shRNA inhibitors useful in the compositions and methods of the instant disclosure are provided in Table 1.

TABLE 1
shRNA Spliceosome Inhibitors

| | | | SEQ |
|---|---|---|---|
| Target* | Sequence | Name | ID NO. |
| U2AF1 | CGGCTGTGATTGACTTGAATAA | sh U2AF1-100 | 1 |
| U2AF1 | ACACCGAGAAAGACAAAGTCAA | shU2AF1-101 | 2 |
| U2AF1 | CCAAAGTCAACTGTTCATTTTA | shU2AF1-102 | 3 |
| U2AF1 | ACTAGAAAGTGTTGTAGTTGAT | shU2AF1-103 | 4 |
| U2AF1 | CCTGCTAGAAAGTGTTGTAGTT | shU2AF1-104 | 5 |
| U2AF1 | ACCTCTTGAACATTTACCGTAA | shU2AF1-105 | 6 |
| U2AF1 | ACTAGAAAGTGTTGTAGTTGAT | shU2AF1-106 | 7 |
| U2AF1 | ACACCGAGAAAGACAAAGTCAA | shU2AF1-107 | 8 |
| PHF5α | CGCCCACTAGTCTCATATTATT | shPHF5α-861 | 9 |
| PHF5α | AGCATTTACTTGTTTAACACTT | shPHF5α-402 | 10 |
| PHF5α | ACTCAACCAAGATCTTCTAAAA | shPHF5α-133 | 11 |
| PHF5α | ACTCTAAGACAGACCTCTTCTA | shPHF5α-103 | 12 |
| PHF5α | ATGGCAAGTGTGTGATTTGTGA | shPHF5α-104 | 13 |
| PHF5α | ATCGGAAGACTGTGTGAAAAAT | shPHF5α-105 | 14 |
| PHF5α | ATAAGACAGACCTCTTCTATGA | shPHF5α-106 | 15 |
| PHF5α | CCGGAGAAAACTTGATAGATTA | shPHF5α-107 | 16 |
| PHF5α | CCCAGGAGTGCCTGCTAGTGTA | shPHF5α-108 | 17 |
| DDX1 | ACACGGTGTTCCTTATGTTATA | shDDX1-301 | 18 |

*KIF11 was used as a positive control to promote cell cycle arrest. The shRNA is named
shKIF11-555, which has the following sequence: ACAAGAGAGGAGTGATAATTAA (SEQ ID NO: 19).

In certain aspects, the present invention includes methods for identifying PHF5α antagonists, U2AF1 antagonists, DDX1 antagonists, or combinations thereof. Methods for identifying the antagonists described herein include any assay that provides for the identification of candidate agents that can affect (e.g., inhibit) expression and/or activity of PHF5α, U2AF1 and/or DDX1.

In one embodiment, the present invention includes a method for identifying a PHF5α antagonist, a U2AF1 antagonist, a DDX1 antagonist. The method can include a) providing at least one candidate agent; b) contacting the at least one candidate agent with proliferating cancer cells having an oncogenic pathway (e.g., an aberrant Ras pathway); c) determining whether the at least one candidate agent inhibits proliferation of the cancer cells, wherein inhibition indicates inhibition of PHF5α, U2AF1, DDX1, or a combination thereof, by the at least one candidate agent, thereby identifying the PHF5α antagonist, U2AF1 antagonist, DDX1 antagonist, or the combination thereof. In some embodiments, the methods can further include determining whether the at least one candidate agent binds to PHF5α, U2AF1, DDX1, or a combination thereof, thereby identifying the PHF5α antagonist, U2AF1 antagonist, DDX1 antagonist, or the combination thereof. In some embodiments, the methods can include identifying PHF5α antagonists. In certain embodiments, the methods can include identifying U2AF1 antagonists. In some embodiments, the methods can include identifying DDX1 antagonists. One of ordinary skill in the art will appreciate that cell proliferation assays are well known in the art as well as suitable assays that can be used to practice the methods provided herein.

In another embodiment, the present invention includes a method for identifying a PHF5α antagonist, a U2AF1 antagonist, a DDX1 antagonist, or a combination thereof, the method including a) providing at least one candidate agent; b) contacting the at least one candidate agent with proliferating cancer cells having an oncogenic pathway (e.g., an aberrant Ras pathway); c) determining whether cell cycle arrest, dysregulated cell cycle progression, dysregulated RNA processing, or a combination thereof is generated in proliferating cancer cells due to inhibition of PHF5α, U2AF1, DDX1, or a combination thereof, wherein cell cycle arrest, dysregulated cell cycle progression, dysregulated RNA processing, or a combination thereof indicates inhibition of PHF5α a, U2AF1, DDX1, or a combination thereof by the at least one candidate agent, thereby identifying PHF5α antagonist, U2AF1 antagonist, DDX1 antagonist, or the combination thereof. In some embodiments, the methods can further include d) determining whether the at least one candidate agent binds to PHF5α, U2AF1, DDX1, or a combination thereof, thereby identifying the PHF5α antagonist, U2AF1 antagonist, DDX1 antagonist, or the combination thereof. In some embodiments, the methods can include identifying PHF5α antagonists. In certain embodiments, the methods can include identifying U2AF1 antagonists. In some embodiments, the methods can include identifying DDX1 antagonists.

In some embodiments, the present invention includes a method of treating a subject having a cellular hyperproliferative disorder associated with an oncogenic pathway, the method comprising administering a pharmaceutical composition comprising a therapeutically effective amount of the PHF5α antagonist, U2AF1 antagonist, DDX1 antagonist, or the combination thereof identified using the methods described herein.

The methods of identifying antagonists can include assays of screening for a candidate agent that can function as an antagonist of, e.g., PHF5α, U2AF1, DDX1, SF3b (a small molecule compound (e.g., a drug), a peptide, or any of the other candidate agents described herein) and identifying an agent for treating a condition or disease associated with an oncogenic pathway (e.g., an aberrant Ras pathway associated, e.g., with a cellular hyperproliferative disorder, such as glioma). A “candidate agent” as used herein, is any substance with a potential to reduce, reverse, interfere with or PHF5α, U2AF1, DDX1, or SF3b expression or activity. Examples of candidate agents include any biologically, physiologically, or pharmacologically active substances that act locally or systemically in a subject. Candidate agents include for example, drugs (e.g. pharmaceutical small molecule compounds) such as those described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or prodrugs, which become biologically active or more active after they have been placed in a physiological environment. Candidate agents also include, for example, small molecules, antibiotics, antivirals, antifungals, enediynes, heavy metal complexes, hormone antagonists, non-specific (non-antibody) proteins, sugar oligomers, aptamers, oligonucleotides (e.g., antisense oligonucleotides that bind to a target nucleic acid sequence (e.g., mRNA sequence)), siRNA, shRNA, peptides, proteins, radionuclides, and transcription-based pharmaceuticals. In some embodiments, candidate agents may be screened by the methods described herein, including nucleic acids, peptides, small molecule compounds (e.g., pharmaceutical compounds), and peptidomimetics. Small molecule compounds (e.g., drugs or pharmaceutical compounds that are organic molecules) can be made using known techniques and further chemically modified, in some embodiments, to facilitate intranuclear transfer to, e.g., the spliceosome. One of ordinary skill in the art will appreciate the standard medicinal chemistry approaches for chemical modifications for intranuclear transfer (e.g., reducing charge, optimizing size, and/or modifying lipophilicity). In the case of peptides, known nuclear translocation sequences can also be used with the peptides to facilitate intranuclear transport. In some embodiments, the peptides can include a nuclear localization signal or sequence (NLS), which is an amino acid sequence that ‘tags’ a protein for import into the cell nucleus by nuclear transport. Classical and non-classical sequences can be used. Peptides that can be screened can have span a range of amino acid lengths, e.g., between 10 to 100 amino acids, or higher.

In certain aspects, candidate agents may be screened from large libraries of synthetic or natural compounds (e.g. pharmaceutical small molecule compounds and/or peptides). One example is an FDA approved library of compounds that can be used by humans. In addition, synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.), and a rare chemical library is available from Aldrich (Milwaukee, Wis.). Combinatorial libraries are available and can be prepared. Alternatively, libraries of natural compounds in the form of bacterial, fungal, plant and animal extracts are also available, for example, Pan Laboratories (Bothell, Wash.) or MycoSearch (NC), or can be readily prepared by methods well known in the art. Compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. It should be understood, although not always explicitly stated that the agent can be used alone or in combination with another modulator, having the same or different biological activity as the agents identified by the subject screening method. Several commercial libraries can immediately be used in the screens.

Candidate agents may include molecules that include, e.g., small peptides or peptide-like molecules (e.g., a peptidomimetic). As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics. In other embodiments, candidate agents also encompass numerous chemical classes, though typically they are organic molecules, often small organic compounds having a molecular weight of more than 50 and less than about 2,500 daltons.

In some embodiments, a library containing a plurality of candidate agents may be evaluated to determine the most desirable candidate agents. For example, such libraries can be generated on the basis, e.g., of binding affinities to PHF5α, U2AF1, DDX1, SF3b and/or ability of the candidate agent to generate a phenotype associated with inhibiting PHF5α, U2AF1, DDX1, or SF3b (e.g., promotes cell cycle arrest, dysregulated cell cycle progression, dysregulated RNA processing, or a combination thereof), and derivatives thereof and modulating activities of PHF5α, U2AF1, DDX1, SF3b and derivatives thereof. Potential lead candidate agents can then be screened in subsequent assays to identify those that display optimal PHF5α, U2AF1, DDX1, SF3b modulating of (e.g., inhibiting of) activity and/or expression.

In some aspects, the methods for identifying antagonists provided herein can include identifying a variety of phenotypes that result from, e.g., inhibition of activity and/or expression of PHF5α, U2AF1, DDX1, and/or SF3b. For example, the methods can include determining whether cell cycle arrest, dysregulated cell cycle progression, dysregulated RNA processing, or a combination thereof is generated due to affecting activity and/or expression of PHF5α, U2AF1, DDX1, SF3b or a combination thereof, wherein cell cycle arrest, dysregulated cell cycle progression, dysregulated RNA processing, or a combination thereof indicates affecting activity and/or expression of PHF5α, U2AF1, DDX1, SF3b or a combination thereof by at least one candidate agent. Determination of the various cell cycle arrest, dysregulated cell cycle progression, and/or dysregulated RNA processing can be performed using any suitable technique known in the art. For example, various staining and/or DNA content analysis methods can be used for determining a cell cycle arrest and/or dysregulated cell cycle progression associated with affecting activity and/or expression of PHF5α, U2AF1, DDX1, SF3b or a combination thereof. As described herein, e.g., MPM-2 staining, indicative of cyclinB/CDK activity, can be used to confirm mitotic arrest of cells (e.g., cancer cells) that are administered a PHF5α, U2AF1, DDX1, and/or SF3b antagonist. DNA content analysis can also be used to identify a percentage of G2/M cells in cells (e.g., cancer cells) that are administered a PHF5α, U2AF1, DDX1, and/or SF3b antagonist. One of ordinary skill in the art will readily appreciate the myriad of other ways that cell cycle arrest can be analyzed. Some example techniques can be found, e.g., in Humphrey and Brooks, Cell Cycle Control: Mechanisms and Protocols: Humana Press (2010), which is incorporated herein by reference.

Identification of dysregulated RNA processing that results from affecting activity and/or expression of PHF5α, U2AF1, DDX1, SF3b or a combination thereof can be determined using a variety of known techniques generally available in the art. Suitable methods can be found, e.g., in the examples provided herein and can include, e.g., splicing reporter assays. In addition, other methods can be modified and used accordingly in view of methods described, e.g., in Younis et al., Mol. Cell. Biol. 30(7): 1718-1728 (2010); Orengo et al., Nucleic Acids Research 34(22) e148 (2006); Nasim and Eperon, Nature Protocols, 1(2): 1022 (2006); Gurskaya et al., Analysis of Alternative Splicing of cassette exons at single-cell level using two fluorescent proteins; Nucleic Acids Res., 1-6 (2012); Xiao et al., Nat. Struct. & Mol. Biol., 16(10):1094-1101 (2009), each of which is incorporated by reference herein.

In some embodiments, the present invention includes methods of treating a cellular hyperproliferative disorder associated with an oncogenic pathway (e.g., an aberrant Ras pathway) combined with methods for identifying PHF5α antagonists, a U2AF1 antagonists, a DDX1 antagonists, or a combination thereof. For example, the present invention includes a method for treating a cellular hyperproliferative disorder associated with an oncogenic pathway (e.g., an aberrant Ras pathway). The method can include a) identifying at least one candidate agent that is a PHF5α antagonist, a U2AF1 antagonist, a DDX1 antagonist, or a combination thereof; b) determining whether a subject is suffering from a cellular hyperproliferative disorder associated with an oncogenic pathway (e.g., an aberrant Ras pathway); and c) if the subject is suffering from a cellular hyperproliferative disorder associated with an oncogenic pathway (e.g., an aberrant Ras pathway), administering the subject in need thereof a therapeutically effective amount of the PHF5α antagonist, the U2AF1 antagonist, the DDX1 antagonist, or the combination thereof. In some embodiments, the methods can include administering and/or identifying PHF5α antagonists. In certain embodiments, the methods can include administering and/or identifying U2AF1 antagonists. In some embodiments, the methods can include administering and/or identifying DDX1 antagonists. One of ordinary skill in the art will appreciate the myriad ways for determining whether a subject is suffering from a cellular hyperproliferative disorder associated with an oncogenic pathway (e.g., an aberrant Ras pathway). For example, a sample (e.g., a tumor sample) can be obtained from the subject an analyzed to determine whether cells with an oncogenic pathway (e.g., an aberrant Ras pathway) are present. In certain embodiments, e.g., the determining can include determining whether the aberrant Ras pathway comprises an aberrantly activated Ras effector. In some embodiments, the determining can include determining whether the aberrant Ras pathway comprises a mutation in RTK, Raf, MAPK, ERK, MEK, MKK, AKT, PI3K, Myc, or any combination thereof. A variety of known techniques (e.g., genetic assays) can be used to, e.g., identify if particular mutations are present.

In some embodiments, the present invention further includes methods for identifying SF3b antagonists. The methods for identifying SF3b antagonists can be based at least in-part on the discovery that 3′ mRNA splice site recognition is disrupted by SF3b antagonists (e.g., Sudemycin C1, pladienolide, or Spliceostatin A) so as to affect viability of cancer cells with inappropriate activity (e.g., aberrant activity) of RTK/Ras, PI3K/AKT, or myc pathways. In addition, it has been further discovered that cancer cells with inappropriate activity of an oncogenic pathway (e.g., aberrant activity of RTK/Ras, PI3K/AKT, or myc pathways) are differentially sensitive to SF3b antagonists (e.g., Sudemycin C, pladienolide, or Spliceostatin A). The present invention includes methods for identifying SF3b antagonists based in-part on these discoveries For example, based in-part on the disruption of 3′ mRNA splice site recognition, the present invention includes a variety of ways for identifying the ability of candidate agents to affect (e.g., inhibit) activity and/or expression of SF3b, thereby allowing for identification of SF3b antagonists. In some embodiments, multiple splicing reporter assays published in the literature which could be adapted for this screen. See, e.g., Younis et al., Mol. Cell. Biol. 30(7): 1718-1728 (2010); Orengo et al., Nucleic Acids Research 34(22) e148 (2006); Nasim and Eperon, Nature Protocols, 1(2): 1022 (2006); Gurskaya et al., Analysis of Alternative Splicing of cassette exons at single-cell level using two fluorescent proteins; Nucleic Acids Res., 1-6 (2012); Xiao et al., Nat. Struct. & Mol. Biol., 16(10):1094-1101 (2009). A de-novo splicing reporter could also be generated either using a system like pSpliceExpress (Kishore et. al. Rapid generation of splicing reporters with pSpliceExpress. Gene. Volume 427, Issues 1-2, 31 Dec. 2008, Pages 104-110) using traditional molecular biology and cloning techniques. Reporter constructs may utilize one or more detectable reporter genes such as, but not limited to, luciferase of a fluorescent protein. The splicing reporter will produce a primary transcript that includes exonic sequences separated by intronic sequence(s) that may be removed from the transcript by cellular splicing machinery. The splicing of the reporter transcript, and consequent inclusion or exclusion of the intervening sequence, can modulate the detectability of the reporter gene, either by completing or disrupting the final protein function. The sequence of the 3′ splice sites would be chosen to best facilitate the screens below.

The screen could be set up to detect at least one of two aspects regarding SF3b inhibition: a) greater splicing perturbation in cancer cells compared to normal cells, or b) reduced recognition of introns with C-rich 3′ splice sites. Splicing perturbation in cancer compared to normal cells would be one method. Normal and cancer cell lines expressing the above reporter constructs would be seeded in multi-well plates and exposed to test compounds for a limited amount of time. Reporter activity would be measured and compared to controls for each cell line, as well as across cell lines to identify compounds that selectively reduce splicing of the reporter gene in the cancer cells compared to the non-cancerous cells. C-rich 3′ splice site recognition would be another method. Reporter constructs and reporter cell lines would be generated that differ in the sequence of the 3′ splice sites within the reporter gene(s). Cells would be screened as above to identify compounds that impede proper splicing of reporter genes containing C-rich splice sites but not canonical splice sites.

In one aspect, the present invention includes a method for identifying a SF3b antagonist. The method can include a) providing at least one candidate agent; b) contacting the at least one candidate agent with proliferating cancer cells; and c) determining whether dysregulated RNA processing is generated in the proliferating cancer cells due to inhibition of SF3b, wherein dysregulated RNA processing indicates inhibition of SF3b by the at least one candidate agent, thereby identifying the SF3b antagonist. In some embodiments, the method can further include determining whether the at least one candidate agent binds to SF3b, thereby identifying the SF3b antagonist.

In another aspect, the present invention includes a method for identifying a SF3b antagonist, the method including a) providing at least one candidate agent; b) contacting the at least one candidate agent with proliferating cancer cells associated with an oncogenic pathway; c) determining whether the proliferating cancer cells are differentially sensitive to the at least one candidate agent, wherein the differential sensitivity indicates inhibition of SF3b by the at least one candidate agent, thereby identifying the SF3b antagonist. In some embodiments, the method can further include d) determining whether the at least one candidate agent binds to SF3b, thereby identifying the SF3b antagonist.

In some embodiments, the present invention includes a method of treating a subject having a cellular hyperproliferative disorder associated with an oncogenic pathway, the method comprising administering a pharmaceutical composition comprising a therapeutically effective amount of the SF3b antagonist identified using the methods described herein. In some embodiments, such SF3b antagonists may be a siRNA, shRNA, miRNA, antisense oligonucleotide, or the like. Alternatively, a SF3b antagonist may be a pharmaceutical compound (or small molecule compound). In some embodiments, the SF3b antagonist promotes cell cycle arrest, dysregulated cell cycle progression, dysregulated RNA processing (e.g., global exon skipping, aberrant constitutive junction splicing, constitutive intron retention), or a combination thereof in glioma cells, but does not have such effects or is minimally active against normal cells.

In some embodiments, the present invention includes methods of treating a cellular hyperproliferative disorder associated with an oncogenic pathway (e.g., an aberrant Ras pathway) combined with methods for identifying SF3b antagonists, or a combination thereof. For example, the present invention includes a method for treating a cellular hyperproliferative disorder associated with an oncogenic pathway (e.g., an aberrant Ras pathway). The method can include a) identifying at least one candidate agent that is a SF3b antagonist; b) determining whether a subject is suffering from a cellular hyperproliferative disorder associated with an oncogenic pathway (e.g., an aberrant Ras pathway); and c) if the subject is suffering from a cellular hyperproliferative disorder associated with an oncogenic pathway (e.g., an aberrant Ras pathway), administering the subject in need thereof a therapeutically effective amount of the SF3b antagonist. One of ordinary skill in the art will appreciate the myriad ways for determining whether a subject is suffering from a cellular hyperproliferative disorder associated with an oncogenic pathway (e.g., an aberrant Ras pathway). For example, a sample (e.g., a tumor sample) can be obtained from the subject an analyzed to determine whether cells with an oncogenic pathway (e.g., an aberrant Ras pathway) are present. In certain embodiments, e.g., the determining can include determining whether the aberrant Ras pathway comprises an aberrantly activated Ras effector. In some embodiments, the determining can include determining whether the aberrant Ras pathway comprises a mutation in RTK, Raf, MAPK, ERK, MEK, MKK, AKT, PI3K, Myc, or any combination thereof. A variety of known techniques (e.g., genetic assays) can be used to, e.g., identify if particular mutations are present.

In some embodiments, the present disclosure provides compositions of a SF3b antagonist, wherein the SF3b antagonist is in a pharmaceutically acceptable diluent, carrier, or excipient.

The present invention also provides methods and compositions for administering the PHF5α, U2AF1, DDX1 antagonists and/or SF3b antagonists described herein to a subject to facilitate diagnostic and/or therapeutic applications. In some embodiments, a subject can include, but is not limited to, a mouse, a rat, a rabbit, a human, or other animal. In certain embodiments, the compositions can include a pharmaceutically acceptable excipient. Pharmaceutical excipients useful in the present invention include, but are not limited to, binders, fillers, disintegrants, lubricants, coatings, sweeteners, flavors and colors. One of skill in the art will recognize that other pharmaceutical excipients are useful in the present invention. The term “pharmaceutical composition” as used herein includes, e.g., solid and/or liquid dosage forms such as tablet, capsule, pill and the like.

The compositions of the present invention can be administered as frequently as necessary, including hourly, daily, weekly or monthly. The antagonists utilized in the methods of the invention can be, e.g., administered at dosages that may be varied depending upon the requirements of the subject the severity of the condition being treated and/or imaged, and/or the antagonist being employed. For example, dosages can be empirically determined considering the type and stage of disease diagnosed in a particular subject and/or the type of imaging modality being used in conjunction with the antagonists. The dose administered to a subject, in the context of the present invention should be sufficient to effect a beneficial diagnostic or therapeutic response in the subject. The size of the dose also can be determined by the existence, nature, and extent of any adverse side-effects that accompany the administration of a particular antagonist (e.g., a PHF5α antagonist) in a particular subject. Determination of the proper dosage for a particular situation is within the skill of the practitioner.

The compositions described herein can be administered to the subject in a variety of ways, including parenterally, intravenously, intradermally, intramuscularly, colonically, rectally or intraperitoneally. In some embodiments, the pharmaceutical compositions can be administered parenterally, intravenously, intramuscularly or orally. The oral agents comprising an antagonist of the invention (e.g., a PHF5α antagonist) can be in any suitable form for oral administration, such as liquid, tablets, capsules, or the like. The oral formulations can be further coated or treated to prevent or reduce dissolution in stomach. The compositions of the present invention can be administered to a subject using any suitable methods known in the art. Suitable formulations for use in the present invention and methods of delivery are generally well known in the art. For example, the antagonists described herein can be formulated as pharmaceutical compositions with a pharmaceutically acceptable diluent, carrier or excipient. The compositions may contain pharmaceutically acceptable auxiliary substances as required to approximate physiological conditions including pH adjusting and buffering agents, tonicity adjusting agents, wetting agents and the like, such as, for example, sodium acetate, sodium lactate, sodium chloride, potassium chloride, calcium chloride, sorbitan monolaurate, triethanolamine oleate, etc.

In certain embodiments, compositions and methods of the instant disclosure could be used to examine a biological sample from a subject having or suspected of having a cellular hyperproliferative disorder (e.g., glioma) by probing for the presence of characteristic dysfunctional RNA splicing (e.g., global exon skipping, aberrant constitutive junction splicing, constitutive intron retention), wherein the probing may comprise contacting a biological sample with an antagonist of PHF5α, U2AF1 or DDX1, SF3b or a combination thereof, wherein the presence of dysfunctional RNA splicing may function as a biomarker for susceptibility of a particular cellular hyperproliferative disorder (or other cell type) to inhibition of PHF5α, U2AF1, DDX1, SF3b or a combination thereof. For example, splice junction primers may be used in digital PCR amplification or quantitative PCR (qPCR) to examine mRNA splice junctions or for the presence or absence of certain exons after a biological sample has been contacted with an antagonist of PHF5α, U2AF1 DDX1, SF3b or a combination thereof. In further embodiments, such compositions and methods of the instant disclosure used to identify the presence of certain biomarkers are used to detect or diagnose the presence of or the risk of having a particular cellular hyperproliferative disorder, having circulating tumor cells, or to assess response to therapy (e.g., response to a therapy targeting PHF5α, U2AF1, DDX1, SF3b or a combination thereof).

Alternatively, compositions and methods of the instant disclosure could be used to quantify proteins that are members of the spliceosome, such as PHF5α, U2AF1, DDX1, SF3b or a combination thereof, as a diagnostic or prognostic test for response to PHF5α, U2AF1, DDX1, or SF3b inhibitor compounds or molecules. In further embodiments, compositions and methods of the instant disclosure could be used to measure activation of Ras/PI3K/Myc pathways in cellular hyperproliferative disorders to screen for or to identify therapeutic PHF5α, U2AF1, DDX1, SF3b inhibitor compounds or molecules. In certain embodiments, such diagnostic assays are performed on blood, serum or other fluids with cell-free mRNA.

In the following description, certain specific details are set forth in order to provide a thorough understanding of various embodiments of this disclosure. However, upon reviewing this disclosure one skilled in the art will understand that the invention may be practiced without many of these details. In other instances, newly emerging next generation sequencing technologies, as well as well-known or widely available next generation sequencing methods (e.g., chain-termination sequencing, dye-terminator sequencing, reversible dye-terminator sequencing, sequencing by synthesis, sequencing by ligation, sequencing by hybridization, polony sequencing, pyrosequencing, ion semiconductor sequencing, nanoball sequencing, nanopore sequencing, single molecule sequencing, FRET sequencing, base-heavy sequencing, and microfluidic sequencing), have not all been described in detail to avoid unnecessarily obscuring the descriptions of the embodiments of the present disclosure. Descriptions of some of these methods can be found, for example, in PCT Publication Nos. WO 98/44151, WO 00/18957, and WO 2006/08413; and U.S. Pat. Nos. 6,143,496, 6,833,246, and 7,754,429; and U.S. Patent Application Publication Nos. U.S. 2010/0227329 and U.S. 2009/0099041.

Various embodiments of the present disclosure are described for purposes of illustration, such as the use of small interfering RNA (siRNA) or short hairpin RNA, but as those skilled in the art will appreciate upon reviewing this disclosure, use with other nucleic acid molecules, antibodies, small molecules, or compounds for inhibiting PHF5α, U2AF1, DDX1, SF3b expression or activity may also be suitable.

Although specific embodiments and examples of this disclosure have been described for illustrative purposes, various equivalent modifications can be made without departing from the spirit and scope of the invention, as will be recognized by those skilled in the relevant art after reviewing the present disclosure. The various embodiments described can be combined to provide further embodiments. These and other changes can be made to the invention in light of the specification.

### EXAMPLES

### Example 1

### Massively Parallel Screen of shRNA Antagonists of Glioblastoma

To identify genes needed for glioblastoma stem cell (GSC) survival but not for normal neural stem cells (NSCs), targeted and genome-wide functional genetic screening was performed.

The targeted screen was performed using shRNA functional genetic screens targeting 1086 DNA binding factors and most of the human genome (˜19,000 genes) in primary GSC tumor isolates and human fetal NSC-CB660 cells. For these screens, genes required for GSC and NSC in vitro expansion in serum-free monolayer culture were assayed (Pollard et al., Cell Stem Cell 4:568, 2009). A single GBM isolate (G166 cells) along with NSC controls were infected with pools of shRNAs (Luo et al., Cell 137:835, 2009; Paddison et al., Nature 428:427, 2004) in triplicate screening populations and expanded in normal conditions for 21 days. Comparisons of shRNA representation over time in GSCs or NSCs using microarrays or deep sequencing revealed shRNAs that became significantly under- or over-represented during expansion. Candidate GSC-specific lethal genes were defined by those shRNAs significantly underrepresented in GSC cultures relative to NSC control cultures. This initial DNA binding factor screen yielded 27 genes as candidate GBM-lethal hits (data not shown). Retests of each hit were performed using multiple single-shRNA viral clones. Validation studies using single clone outgrowth or mixed in vitro competition with FACS analysis revealed PHF5α knockdown as the top hit affecting in vitro expansion of GSC-G166 cells. Three additional targets were also validated (although these were not as effective as PHF5α)—ZNF207, POLR21, and TFCP2L1. (data not shown)

To ensure that the results were applicable to other GBM tumors and that hits would score similarly when comparing the entire genome, a genome-wide screen was performed in three GSC cultures (G166, 0827, and 0131) and one NSC (CB660) primary cell culture. A massively parallel screen using short hairpin RNAs (shRNAs) for stable loss-of-function phenotypes and array analysis was performed as previously described (Paddison et al., 2004; Silva et al., Science 319:617, 2008; which methods are hereby incorporated by reference). Cells were infected with a pool of pGIPZ lentiviral shRNAs (SIDNET; Open Biosystems, Huntsville, Ala.) targeting a set of human transcription factors or the whole genome at a representation of approximately 1,000 fold (multiplicity of infection, MOI<1) and selected with puromycin to remove uninfected cells. Cells were then propagated in culture for 21 days and sampled for analysis. For each passage, a minimal representation of 1000 fold was maintained. For each corresponding sample, shRNA barcodes were recovered from genomic DNA samples by PCR, and either sequenced using an Illumina Genome analyzer IIx or labeled with Cy5 or Cy3, and competitively hybridized in a microarray containing the corresponding probes (Agilent Technologies, Santa Clara, Calif.; see also Paddison et al., 2004). Massively parallel DNA sequencing, array hybridization, and statistical analysis were carried out and data identifying promising hits were validated in a single-well format using additional shRNA viral clones targeting each gene.

Gene ontology analysis of hits strongly implicated members of the pre-mRNA splicing complex as differentially detrimental to the survival of GSCs as compared to the NSC controls. In particular, many hits from the genome-wide screens identified members of the U2 small nuclear ribonucleoprotein (snRNP) subcomponent of the pre-mRNA splicing complex, but the strongest correlation (with a hit scored in all three GSC cultures tested) was the gene encoding PHF5α. In addition to PHF5α, there were 16 other candidate lethal genes shared by each of the three GSC isolates tested (that did not affect NSCs)—ARL6IP1, C3orf67, CLSTN1, EIF2S1, INTS4, KPNB1, LSM6, PHLDB1, POLR2E, PSMC5, RRM1, RRM2, SNORA21, TRA2B, TRIP13, and VCP.

This indicates that a therapeutic strategy targeting specific members of the RNA splicing machinery may have a therapeutic window in the treatment of gliomas. Identifying genes which when inhibited affect growth of GSCs more than NSCs is unusual. Most perturbations in pathways required for cell growth or cell cycle progression (e.g., PI3K pathway, Aurora A and B kinases, heat shock protein 90, and the microtubule motor protein KIF11/Eg5 (Ding et al., 2012) (data not shown)) either show no differential effect between NSCs and GSCs or effect NSCs more than GSCs.

### Example 2

### PHF5α Knockdown Inhibits Glioma Proliferation

To verify the robustness and reproducibility of PHF5α loss in GSC growth inhibition, the normal NSC control cells and a panel of five GSC primary cell cultures were infected with viruses encoding three different shRNA sequences targeting PHF5α (shPHF5α-133 (SEQ ID NO.:11), shPHF5α-402 (SEQ ID NO.:10), and shPHF5α-861 (SEQ ID NO.:9)), as well as a positive control (shKIF11-555 (SEQ ID NO.:19); blocking kinesin family member 11 (KIF11) prevents centrosome migration, which arrests cells in mitosis) and negative control (shCtrl). Cultures were selected with puromycin, plated at about 1000 cells per well in 96-well plates, and incubated for one week at 37° C./5% CO₂ before assaying cell viability using the alamarBlue® assay (Invitrogen, Gand Island, N.Y.). Briefly, resazurin (a blue compound that is non-toxic, cell permeable compound and essentially non-fluorescent) is added to the cell cultures, which is reduced to resorufin upon entering metabolically active cells (in contrast, non-viable or damaged cells cannot do this effectively). Resorufin is a bright red fluorescent molecule that functions as a colorimetric indicator. In addition, all three shRNA viruses were internally validated to knock down PHF5α at both the RNA and protein levels (data not shown).

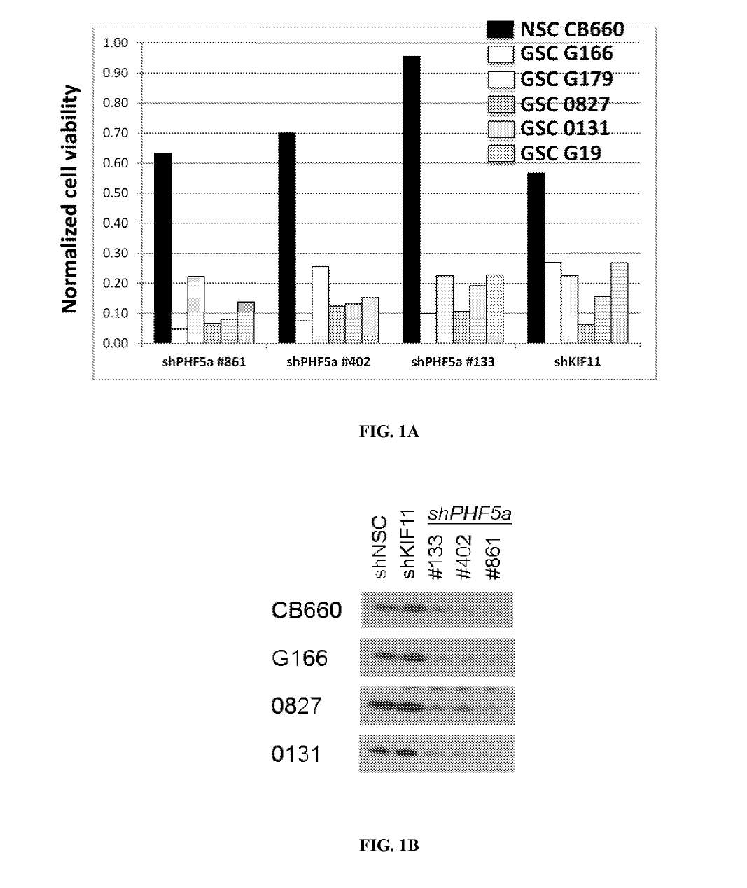

In every case, PHF5α inhibition resulted in a much greater inhibition of GSC growth than NSC growth (see FIGS. 1A and 1B). NSC CB660 cells are shown in FIG. 1A. Similar viability results was also found with untransformed IMR90 fibroblast cells (data not shown). This confirms that the effects seen from shPHF5α viral infection result from PHF5α loss and are not due to a sequence-based off-target effect on an unknown protein. The reproducibility in all five GSC cell cultures also indicates that PHF5α inhibition can effectively kill a range of gliomas.

In addition, the GSC and NSC containing two of the shRNA sequences targeting PHF5α, KIF11 (positive control), and a random clone (negative control) were examined by immunofluorescence analysis using DAPI staining and a fluorescent microscope. Cells that appeared balled up are ones that suffered cell cycle arrest, while elongated cells were unaffected in their proliferation. Knockdown of KIF11 showed mitotic arrest in both GSCs and NSCs, while the random control clone had no effect on any cell line (data not shown). In contrast, inhibition of PHF5α produced a striking phenotype of a G2/M mitotic arrest in GSCs, but not in NSCs (data not shown).

Finally, further examination of mitotic spindle formation indicated that shPHF5A in GSCs, but not NSCs, showed condensed chromatin but primarily formed monopolar or multipolar spindles with few metaphase or anaphase cells (data not shown). These results indicate that PHF5α activity in GSCs, but not NSCs, is required for progression through mitosis.

The effects of PHF5α KD were also examined on SSEA1+GSC subpopulations, which are enriched for tumor-initiating cell activity (Son et al. 2009). In the three different GSC isolates examined, PHF5α KD compromised outgrowth of SSEA1+ populations over the course of several weeks (data not shown). This indicates that PHF5A suppression blocks gross expansion of GSC isolates and tumor-initiating GSC subpopulations.

GSCs and NSCs express PHF5A at relatively similar levels, and KD is equivalently effective in each cell type at both the RNA and protein levels (data not shown), indicating that the lack of phenotype in NSCs is not due to inefficient knockdown or major differences in expression. Moreover, PHF5A expression levels were similar in GSCs, NSCs, and in other tissues, indicating that GSCs do not abnormally overexpress the gene (data not shown). A complementation assay was also performed in which a validated, inducible shPHF5A sequence targeting the PHF5A endogenous 3′UTR was co-expressed with the PHF5A open reading frame (ORF) lacking its endogenous 3′UTR. Expression of the PHF5A ORF rescued the growth defect observed in PHF5A KD GSCs (data not shown), indicating that the phenotypic effects are PHF5A-specific.

To further query what key roles PHF5A might play in our cells, PHF5A-interacting proteins were examined by co-IP mass spectrometry. This yielded a strong enrichment for candidate interacting proteins involved in splicing (GO:0008380 RNA splicing p=10⁻¹⁴), as well as gene expression (GO:0010467 gene expression p=10⁻¹⁶) (data not shown).

### Example 3

### PHF5α knockdown inhibition of glioma proliferation due to spliceosome activity

Ryzinski et al. (Cytogenet. Genuine Res. 121:232, 2008) identified PHF5α as a bridge protein capable of connecting members of the U2 snRNP, especially U2AF1 to RNA helicases, especially DDX1. To test whether, out of the possible known or unknown roles in a cell, the splicing functions of PHF5α is an important activity contributing to GSC survival and proliferation, shRNA knockdown similar to Example 2 was performed against each member of this bridging interaction—U2AF1 and DDX1.

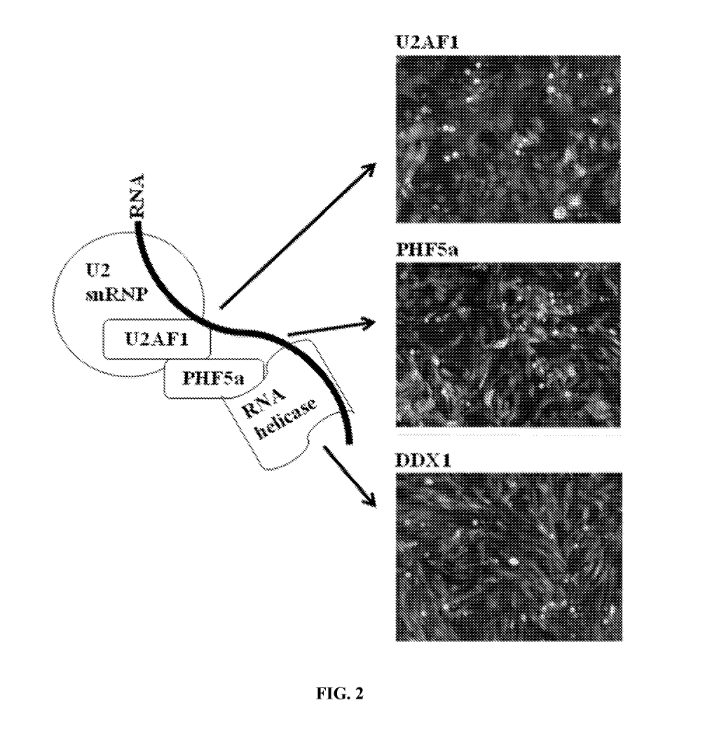

The G2/M cell cycle arrest phenotype caused by PHF5α inhibition was again observed when either U2AF1 or DDX1 were inhibited (see FIG. 2). Again, this arrest only occurred in GSCs (FIG. 2) and not normal NSCs (data not shown). Moreover, comprehensive examination of multiple shRNAs against PHF5α and U2AF1 in short-term growth assays showed the same strong trend of requirement in GSCs versus NSCs (data not shown). Finally, examination of PHF5α interacting proteins by co-immunoprecipitation mass spectrometry yielded strong enrichment (GO:0008380 RNA splicing p=10⁻¹⁴) for candidate interacting proteins involved in splicing and the U2 snRNP complex, including U2AF1, U2AF2 and multiple DDX/DHX helicase family members (see Table 2).

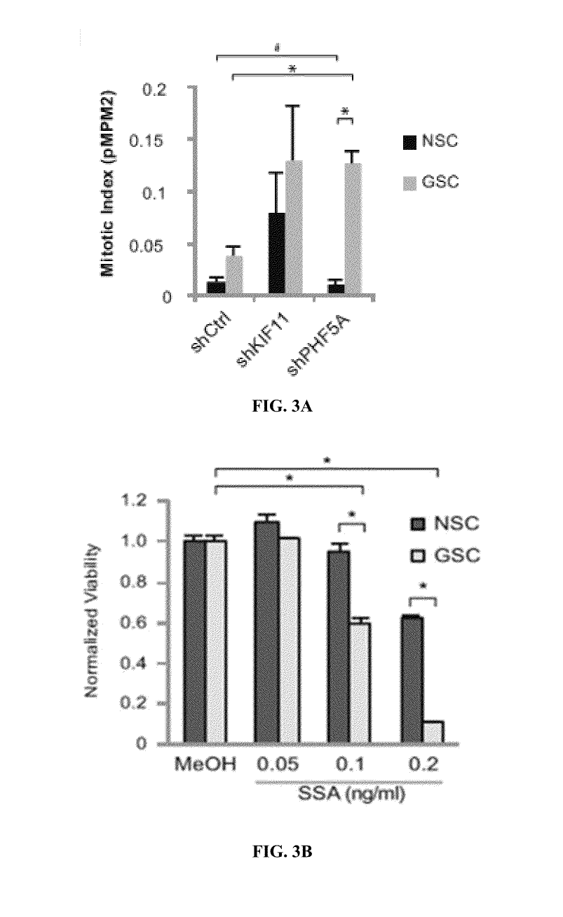

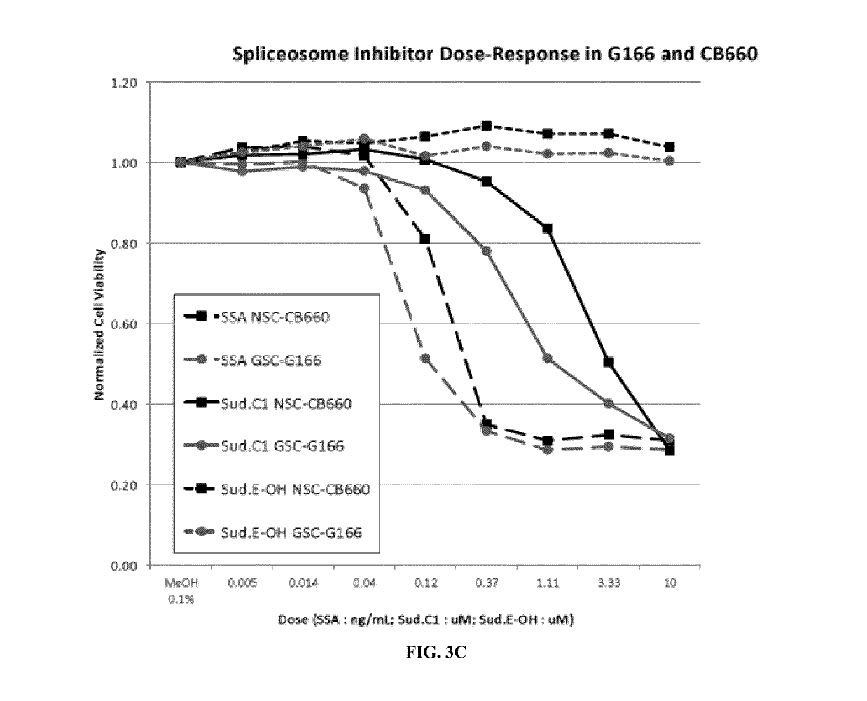

One feature of PHF5A depletion in GSCs was that, preceding widespread GSC cell death, PHF5A KD triggered a dramatic cell cycle arrest that resembled the rounded-up phenotype of kinesin motor protein KIF11 KD (Sawin et al. 1992), our non specific cell-lethal control (data not shown). MPM-2 staining, indicative of CyclinB/CDK activity, dramatically increased in PHF5A KD GSCs, confirming mitotic arrest (FIG. 3A). Moreover, DNA content analysis showed a pronounced increase in the percentage of G2/M cells in GSCs, but not NSCs or normal fibroblasts, with PHF5A KD (data not shown).

Further examination of GSC PHF5A KD G2/M arrested cells showed condensed chromatin and monopolar or multipolar spindles (data not shown). Along with high MPM-2 staining, and little or no phosphorylated BubR1, this is consistent with a pre-anaphase arrest in which the mitotic checkpoint has not been triggered. Consistent with requirement for U2snRNP activity, treatment of GSCs with SSA or SudC1 resulted in a greater dose-dependent viability loss in GSCs relative to NSCs (FIGS. 3B and 3C) and also resulted in the characteristic cell cycle arrest in GSCs but not NSCs at doses within this efficacy window (data not shown).

The GSC-specific G2/M arrest was characterized by performing metaphase capture assays in H2B-GFP expressing GSCs treated with proteasome inhibitor MG132, which arrests mitotic cells at metaphase, blocking APCCdc20-dependent degradation of Cyclin B (Lampson and Kapoor 2005). After overnight exposure to SudC1 or SSA, cells were treated with MG132 for 2 hours. Control cells displayed proper enrichment for metaphase cells, with chromosomes aligned along the metaphase plate (data not shown). However, SSA- or SudC1-treated cells were unable to properly arrest, further suggesting a pre-metaphase arrest (data not shown). Similarly live cell imaging of GSC-H2B-GFP cells treated with SudC1 or SSA treatment showed mitotic arrest pre-metaphase (data not shown). It was also observed that the viability loss in drug-treated GSC cultures results from the death of previously arrested mitotic cells and not interphase GSCs, identifying the cancer-specific mitotic arrest as a causative event in cancer cell death due to splicing inhibition. A fraction of arrested GSCs were able to survive by progress through mitosis after arresting, but these cells displayed disorganized, multi-lobed nuclei and were not observed to successfully divide again (data not shown).

Taken together, the above results establish that PHF5A and U2 snRNP complex activity are differentially required for GSC viability compared to NSCs, and their activity is necessary for GSC but not NSC transit through pre-metaphase mitosis. Moreover, because treatment of GSCs with SSA or SudC 1 did not affect the timing of mitoses for several hours after drug treatment (data not shown), it is unlikely that PHF5A and U2 snRNP activity are directly required for mitotic progression.

This confirms that PHF5α's involvement in the spliceosome is important in its cancer-specific effects.

TABLE 2
Co-Immunoprecipitation-MS Results

| | | | |
|---|---|---|---|
| ABCF1 | AP3D1 | ARGLU1 | BUD13 |
| C1orf35 | C9orf114 | C9orf86 | CCDC49 |
| CCDC55 | CCNL1 | CPSF1 | CPSF2 |
| CPSF3 | CPSF6 | CRKRS | CROP |
| CWF19L2 | DDX17 | DDX18 | DDX27 |
| DDX3X | DDX41 | DDX46 | DDX5 |
| DHX15 | DHX35 | DHX8 | EEF1A1 |
| EFTUD2 | EIF5B | ENO1 | FAM133B |
| FAU | FIP1L1 | FRG1 | GNL3 |
| GPATCH1 | HNRNPH1 | HSPA8 | HSPA9 |
| KRT1 | KRT10 | KRT16 | KRT2 |
| KRT9 | LTV1 | LUC7L2 | LYAR |
| MED19 | MORG1 | NKAP | NOL10 |
| NOP56 | NOP58 | NUDT21 | PARP1 |
| PHF5A | PPIB | PPIL4 | PRDX1 |
| PRPF38B | PRPF40A | PRPF4B | PRPF8 |
| PUF60 | RBBP6 | RBM25 | RBMX2 |
| RCC2 | RP9 | RPL17 | RPL18 |
| RPL27A | RPL32 | RPL35 | RPS23 |
| RPS3 | RPS4X | RPS8 | SAP30BP |
| SART1 | SFRS11 | SFRS12 | SFRS17A |
| SNRNP200 | SNRNP40 | SON | SRPK1 |
| SRRM1 | SRRM2 | TAF2 | TAF3 |
| TAF4 | TAF5 | TAF6 | TAF9 |
| TCOF1 | TINP1 | TOP1 | TRIM21 |
| TUBB2C | TWISTNB | U2AF1 | U2AF2 |
| WDR33 | ZCCHC17 | | |

### Example 4

### PHF5α Knockdown Inhibits Proliferation of Glioma CD15+ Subpopulation

A glioblastoma tumor or primary culture is believed to have only a subpopulation of tumor-initiating cells, which can be identified by expression of the cell surface protein CD15. That is, only the CD15+ cell can fully reform the growth and diversity of the original tumor. Thus, in theory, a successful therapy should be able to destroy these tumor-initiating cells to fully put an end to a tumor. To examine whether PHF5α inhibition affects the CD15+ subpopulation of cells, a mixed cell competition assay was performed. Two GSC cell lines (G166 and 0827) were independently infected with either green fluorescent protein (GFP)-expressing shRNA virus targeting PHF5α or with a non-silencing control (shCtrl). The infected cells were mixed with approximately 20% uninfected cells and then incubated at 37° C./5% CO₂ for 3 weeks. At several time points (day 4, 7, 14, and 21), the ratio of GFP+(infected) cells to the GFP-(non-infected) cells were measured by FACS analysis.

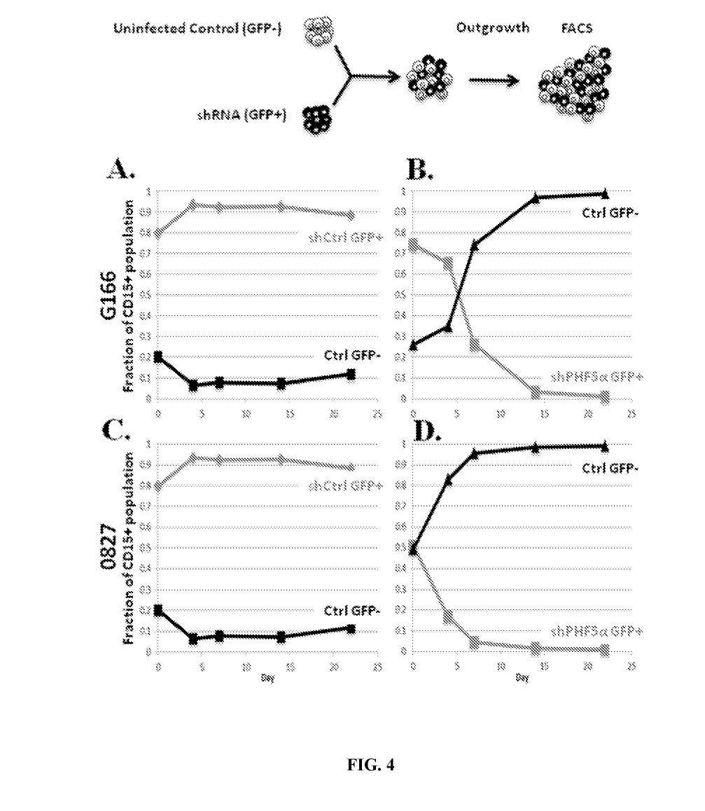

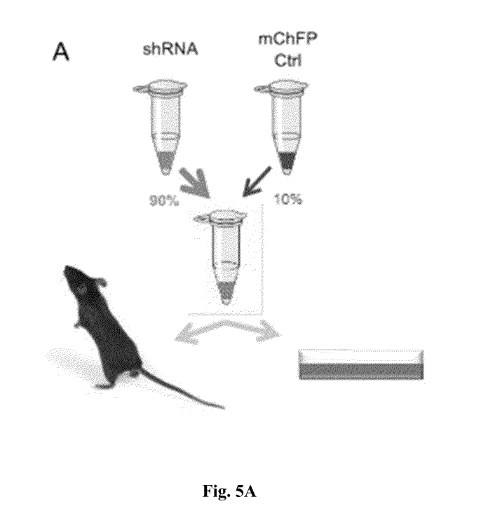

The shCtrl treated cells were able to grow at the same rate as the uninfected cells and maintain their abundance in the mixed population (data not shown). In contrast, the shPHF5α treated cells were not able to grow well and eventually dropped out of the mixed population (data not shown). Importantly, at each time point, an antibody specific for CD15 was used to mark and independently study the response of the tumor-initiating cell subpopulation in each culture. Like in the bulk cell population, CD15+ shCtrl treated cells were able to maintain their growth in the population over the 3 weeks (FIGS. 4A and 4C), but CD15+ shPHF5α-infected cells were rapidly outcompeted by the uninfected cells (FIGS. 4B and 4D). This shows that targeted knockdown of PHF5α affects both the majority of GSCs, as well as the CD15+ subpopulation of tumor-initiating cells.

### Example 5

### PHF5α Knockdown In Vivo Inhibits Glioma Xenograft

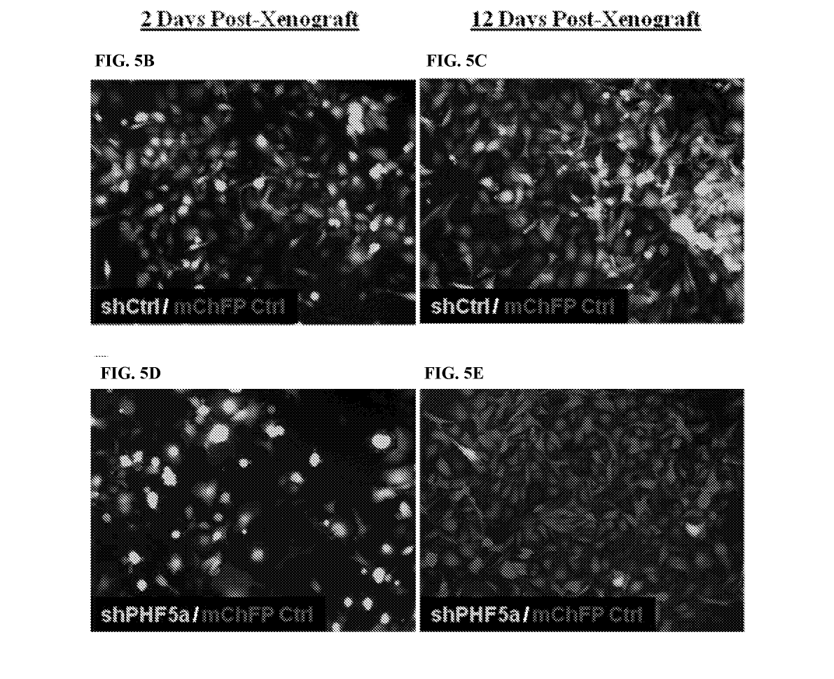

To examine whether PHF5α inhibition is effective in vivo, orthotopic brain xenografts of GSC primary cultures in mice were used for competition experiments. GSC 0131 primary culture cells were infected with GFP-expressing shPHF5α, GFP-expressing shCtrl (green), or mCherry fluorescent protein (mChFP) expressing control virus (red—as an independent control for engraftment and growth rate of each individual mouse xenograft). The cells were mixed at a ratio of 90% green to 10% red cells and about 0.2×10⁶ cells of this mixed population were injected into the right cortex of each mouse brain (see FIG. 5A). Remaining cells were re-plated and incubated in culture at 37° C./5% CO₂ (see FIG. 5A).

Two days after culturing, both the shCtrl and shPHF5α cells were alive and attached to the culture plate surface, which shows that the cells were alive and viable when injected into the mouse brain (see FIGS. 5B and 5D). After 12 days in culture, only the shCtrl cells were able to maintain their 90% representation in culture (see FIG. 5C), whereas the shPHF5α cells were unable to outgrow and were replaced by the mChFP control cells in culture (see FIG. 5E).

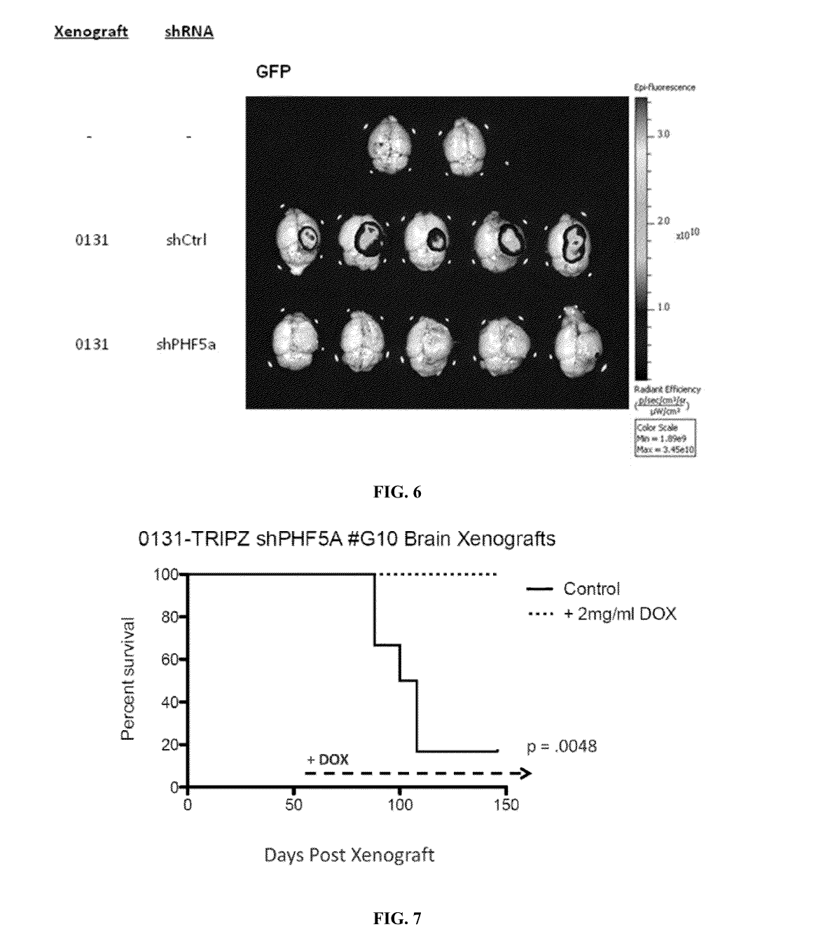

The xenograft mouse tumors were allowed to grow for approximately 5 weeks, at which time the brains were harvested and fluorescently imaged using a Xenogen® IVIS® imaging system (see FIG. 6). Similar to the results seen with the cultured cells, the shCtrl cells were able to proliferate and contribute to the bulk tumor mass as seen by the presence of fluorescence (see FIG. 6, middle row), whereas the shPHF5α treated cells were not detectable (see FIG. 6, bottom row), but still had some tumor load due to the presence and proliferation of the mChFP control cells (data not shown). The control brains treated with vehicle only had no tumor load (see FIG. 6, top row). The small fraction of co-injected ChFP+ control GSCs were able to engraft and give rise to tumors in every case, and ChFP expression mirrored bulk tumor mass as marked by the Chlorotoxin:Cy5.5 conjugate Tumor Paint (Veiseh et al. 2007). This underscores that expression of PHF5A shRNA is a key determinant in whether GSCs contribute to tumor growth. This demonstrates that inhibition of PHF5α prevents glioblastoma tumor engraftment, growth or both in vivo.

In a further experiment in mice having glioblastoma brain xenografts, a Kaplan-Meier analysis of mouse survival was performed in mice containing a 0131 GBM cell clone (designated clone #G10) harboring a doxycycline-inducible shPHF5α-861 shRNA sequence. After the first symptom of tumor presence was detected (after day 50), mice were randomized into two groups with one group given the inducing agent doxycycline (2 mg/ml) in their drinking water.

The Kaplan-Meier survival plot (FIG. 7) shows that the mice given doxycycline (i.e., shPHF5α is produced) all survived, while all but one mouse not having shPHF5α died within 150 days (and the last mouse is moribund). These data show that a PHF5α antagonist is therapeutically effective.

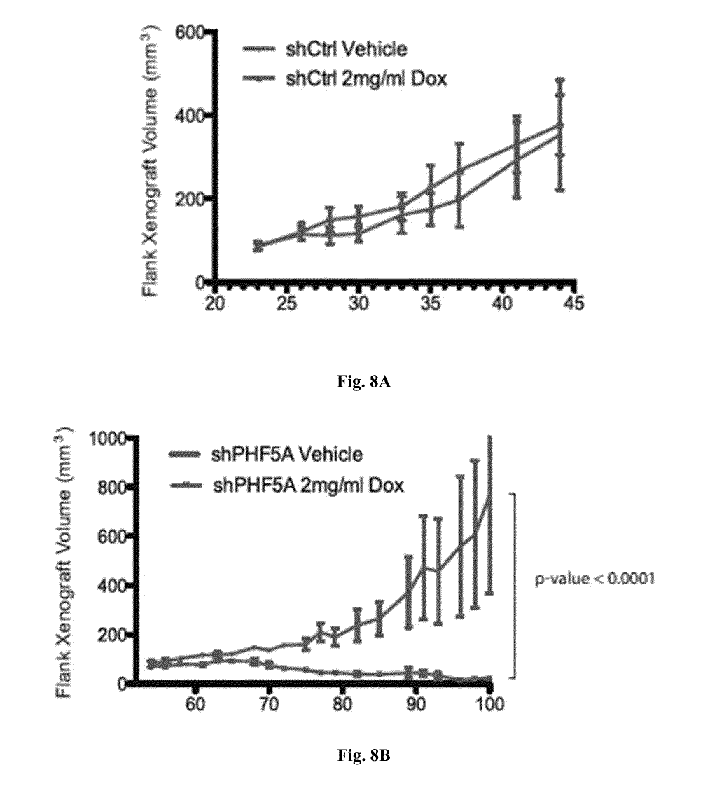

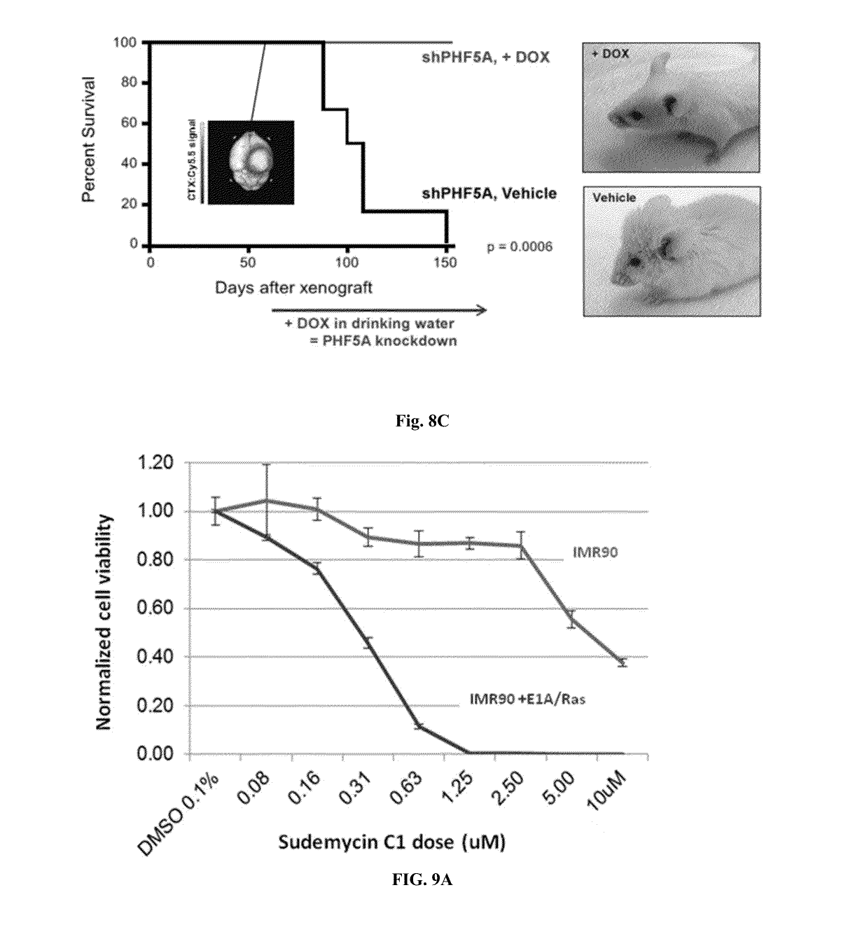

It was also examined whether PHF5A inhibition in established tumors could compromise tumor maintenance, a metric in evaluating potential therapeutic avenues. To this end, xenograft mice bearing GSC tumors with Doxycycline (Dox)-inducible PHF5A shRNA (data not shown) or Ctrl shRNAs were generated. Tumors were allowed to grow to approximately 75 mm³ in size prior to the start of continuous Dox treatment. Whereas control shRNA tumors showed no measurable difference in growth rate upon Dox treatment (FIG. 8A), shPHF5A tumor growth arrested upon Dox administration, and tumors diminished until they were nearly undetectable (FIG. 8B).

It was tested whether brain-derived, GSC-driven tumors would respond to PHF5A suppression. To test this, GSCs bearing Dox-inducible PHF5A shRNA were xenografted into the right cortex of immunocompromised mice. After 52 days, the first mouse showed initial mild symptoms of a brain tumor. CTX:Cy5.5 imaging after sacrifice confirmed a tumor signal in the right cortex (FIG. 8C, inset). The remaining mice were therefore randomized into Dox-treated and vehicle-control cohorts and their survival was followed over time. Survival was significantly improved by PHF5A suppression in the Dox-treated cohort (p=0.0006) to the point where, at the conclusion of the study when all vehicle-treated mice had succumbed to their tumors, 100% of Dox-treated mice were alive and free of symptoms (FIG. 8C). This concludes that PHF5A inhibition compromises both GBM tumor formation and maintenance, indicating that PHF5A/U2snRNP inhibition can be an effective therapy for GBM.

### Example 6

### Effect of PHF5α knockdown sensitized by Ras mutation

Approximately 88% of clinical glioblastoma tumors harbor mutations that activate signaling through the Ras/PI3-kinase pathways (The Cancer Genome Atlas Research Network, Nature 455:1061, 2008). Since PHF5α inhibition is detrimental to a range of glioblastoma cell cultures, but not to the normal NSC from which most glioblastomas are thought to arise, transformation from a normal cell to a cancer cell was examined to determine whether PHF5α dependence was acquired during this process. Accordingly, Ras signaling was activated in normal IMR90 fibroblast cells by infecting with a retrovirus encoding the ElA and Ras oncogenes (EIA was included because Ras alone would be lethal in these cells). These partially transformed fibroblasts were probed for their response to shPHF5α inhibition and to a U2 snRNP splicing inhibitor (sudemycin C1). Viral shRNA knockdown was performed as described in the above examples. For drug response assays, cells were plated in 96-well plates and contacted with a range of sudemycin C1 concentrations (0.08, 0.16, 0.31, 0.63, 1.25, 2.50, 5.0 or 10.0 μM) for a period of 24 hours. Media was then removed and replaced with fresh media, then cell viability was measured 72 hours after drug exposure using the alamarBlue® assay (Invitrogen).

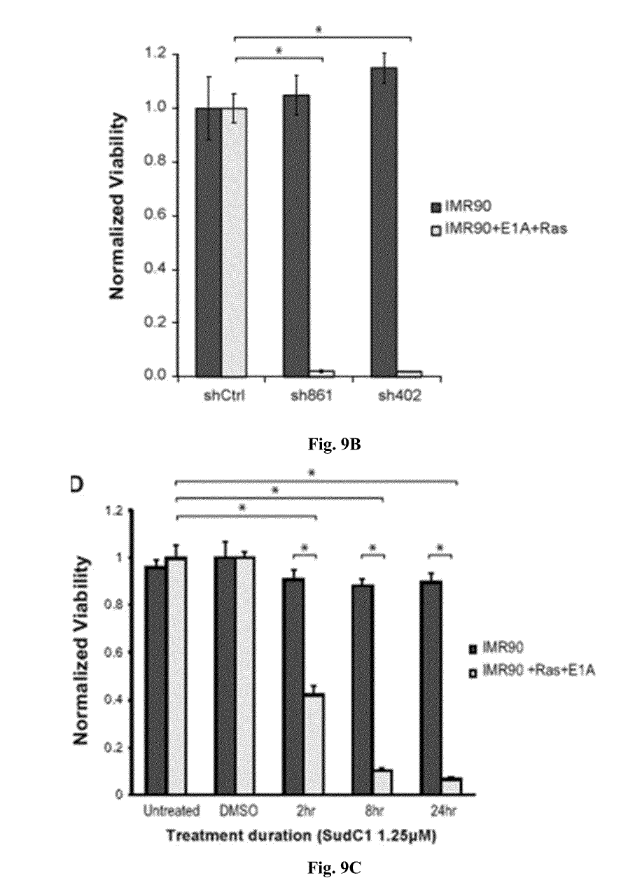

Non-transformed (normal) IMR90 fibroblast cells were relatively resistant to both PHF5α knockdown and to sudemycin treatment, whereas the same cells with the addition of oncogenic Ras signaling were greatly sensitized to the same treatments (see FIG. 9A). FIG. 9A depicts the dramatic sensitivities that occurred in IMR90-Tert fibroblasts partially transformed with RasV 12 and ElA, which are p53 positive. In these cells, PHF5A KD (FIG. 9B) or drug treatments (FIG. 9C) resulted in massive cell death, even after short drug exposure. The figures depict the effect of Ras signaling activation in the MEFs. Non-transformed MEF cells were relatively resistant to both PHF5α knockdown and to sudemycin treatment, whereas the same cells with the addition of oncogenic Ras signaling were greatly sensitized to the same treatments. Similar results were observed in primary human foreskin fibroblasts with the addition of Myc (data not shown), and in mouse embryonic fibroblasts that were p53 deficient (data not shown). Similarly, the RasV 12 mutation expressed in a colorectal cancer cell line (Luo et al., 2009) were screened with PHF5α and U2AF1 shRNAs. Knockdown of either PHF5α or U2AF1 differentially inhibited RasV12 expressing populations (data not shown).

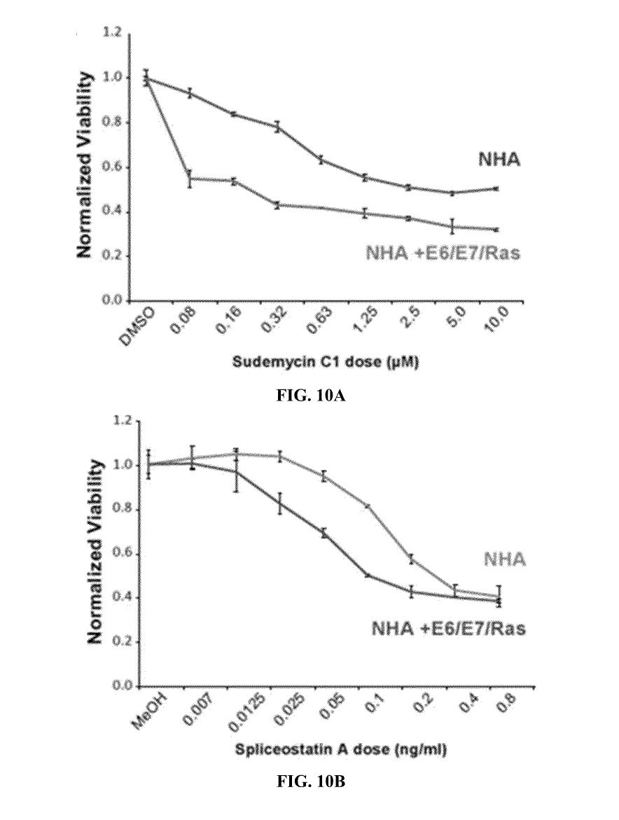

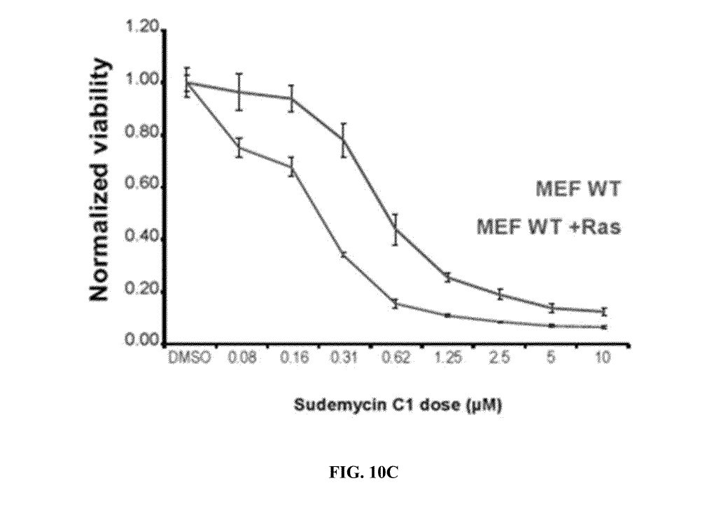

In addition, Ras signaling was activated in normal human astrocytes (NHAs) (Sonoda et al. 2001) as model normal cell systems. The partially transformed fibroblasts were probed for their response to shPHF5α inhibition and to a U2 snRNP splicing inhibitor (sudemycin C1). Viral shRNA knockdown and drug response assays were performed as described in the above examples. RasV12 E6/E7 NHAs showed the same differential sensitivity and characteristic cell cycle arrest accompanied by cell death that was observed in GSCs when treated with PHF5A KD (data not shown) or splicing inhibitors (FIG. 10A-C).

In a separate experiment, drugs Spliceostatin A, Pladienolide B. Clotrimazole, chlorhexidine, TG003 and flunarizine which are identified in literature as exhibiting some degree of splicing inhibition were tested. See FIG. 11. Cells expressing Ras were differentially sensitive to Spliceostatin A, Pladienolide B, and Sudemycin C1, all of which target the SF3b complex (7 protein members including PHF5A). Although the mechanisms of splicing inhibition by the other four drugs, Clotrimazole, chlorhexidine, TG003 and flunarizine, are not well characterized, none of these are believed to target the SF3b complex.

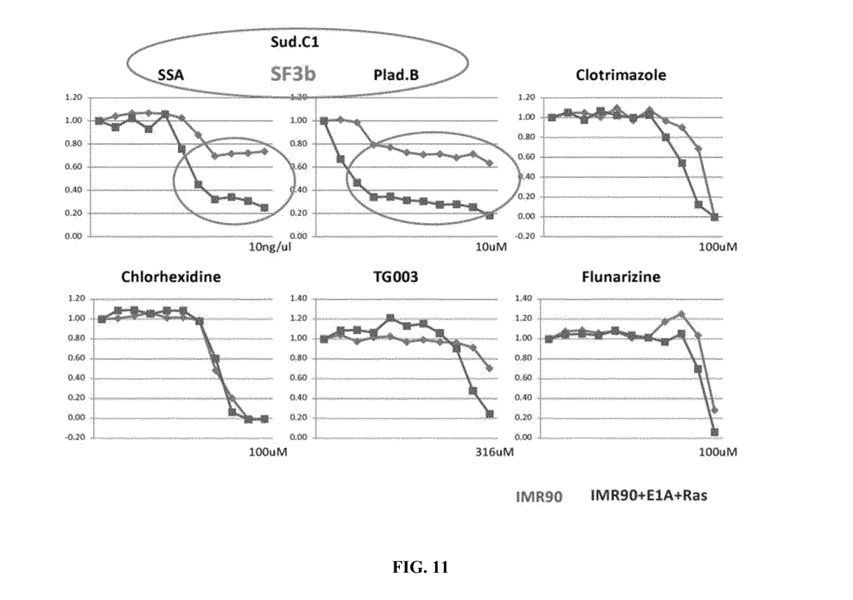

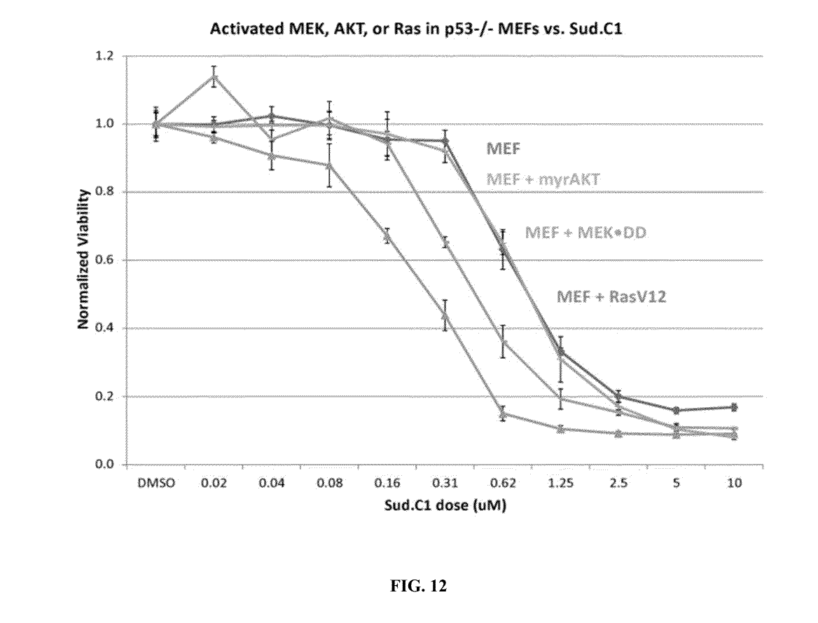

Expression of activated MEK (downstream of Ras in the signaling cascade) partially duplicates the splicing inhibitor sensitivity seen in cells expressing oncogenic Ras. See FIG. 12. This means that Ras pathway activation, not just Ras mutation, can cause splicing inhibitor sensitivity. More generalizable to cancers like GBM where the pathway is frequently active but Ras mutations are rare. Thus, in all systems RasV 12 activity evoked sensitivity to PHF5A and/or U2snRNP inhibition.

This indicates that cancers driven by oncogenic Ras/PI3K pathway signaling or Myc activity are very sensitive to inhibition of proper PHF5α expression or SF3b/U2 snRNP function. Furthermore, these data indicate that oncogenic activity of Ras in other cancers, whether arising through RTK stimulation or Ras mutation, can trigger susceptibility to PHF5α or U2snRNP inhibition.

### Example 7

### PHF5α Knockdown Induces Dysregulated RNA Processing

To identify the changes in pre-mRNA splicing after PHF5α knockdown, RNA sequencing was performed on cultures after shCtrl or shPHF5α infection. GSC and NSC primary cultures were infected with pGIPZ lentiviral shRNAmir vectors (SIDNET; Open Biosystems, Huntsville, Ala.) containing shCtrl or shPHF5α as described in the examples above and selected with puromycin. Cells were harvested and lysed in TRIzol® (Invitrogen) and RNA was isolated according to the manufacturer's instructions. cDNA library sequencing (RNA-seq) was performed using an Illumina Genome Analyzer IIx. Computational analysis was similar to that described in Katz et al. (Nature Methods 7:1009, 2010), but with modifications to obtain single read data.

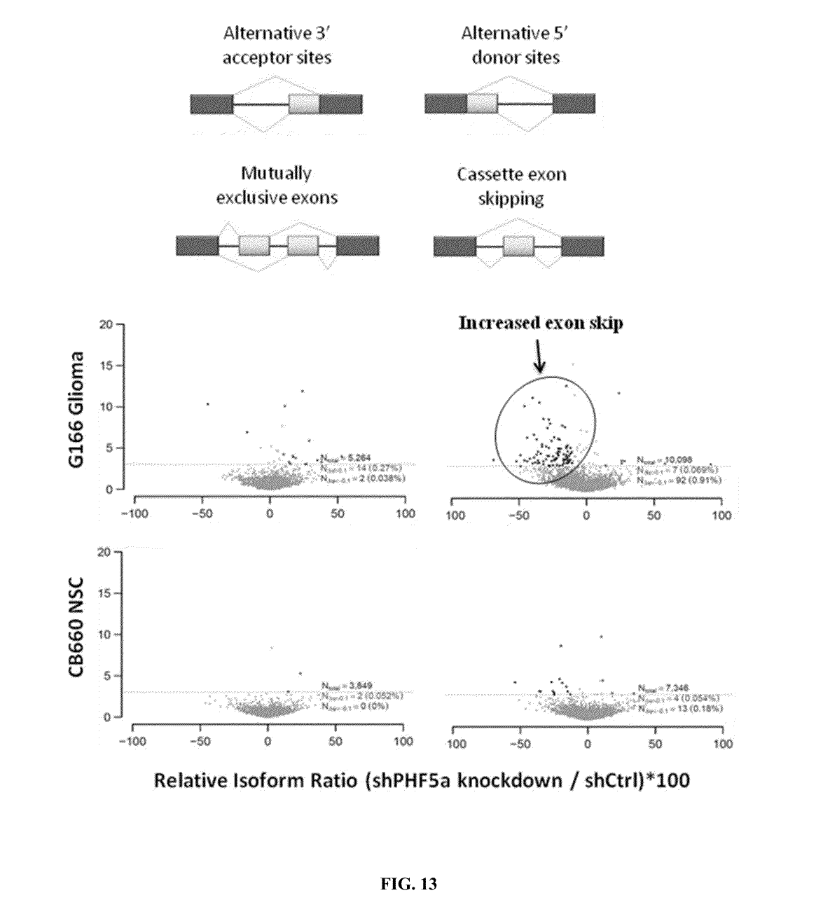

Statistical analysis of the relative abundance of RNA isoforms in shPHF5α knockdown cells compared to shCtrl cells demonstrated that there was a very significant change in the frequency of certain splicing events in cancer, but not in normal, cells. Initial findings showed that after PHF5α inhibition, GSC cells exhibited a strong global trend towards exclusion of cassette exons from their mature RNA transcripts, resulting in an altered ratio of protein isoforms in the cancer cell (see FIG. 13). Importantly, as discussed below, later data extended these results from skipping of cassette exons to a broad failure to properly recognize constitutive exons and introns.

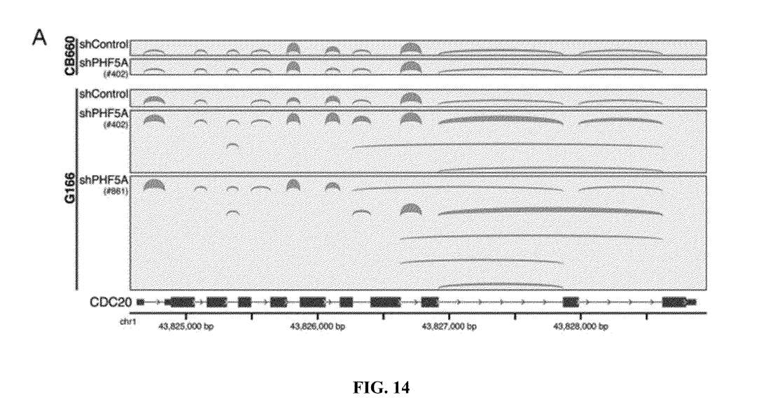

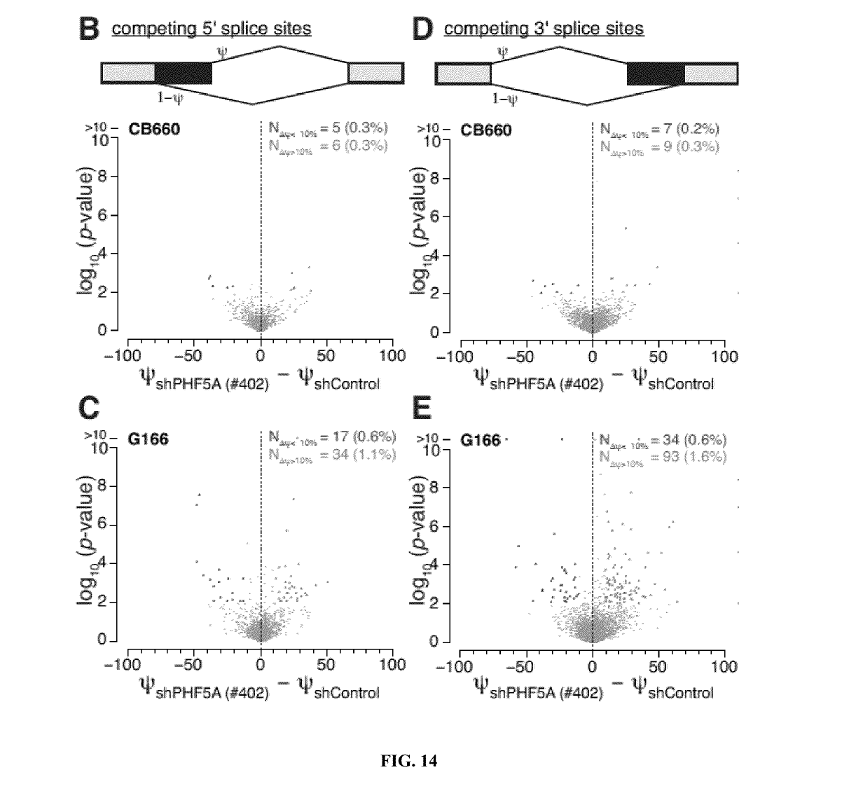

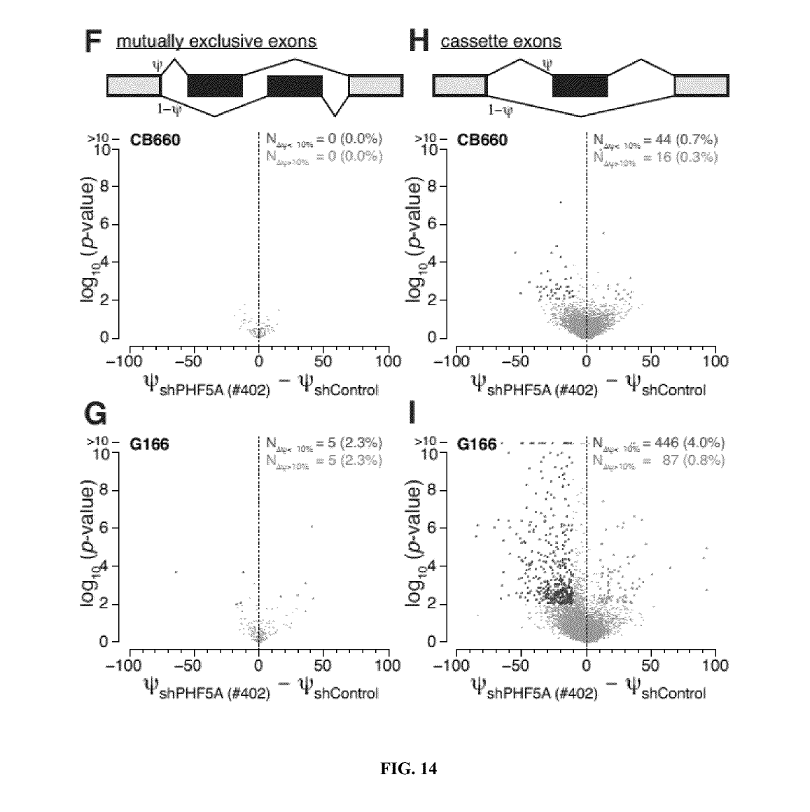

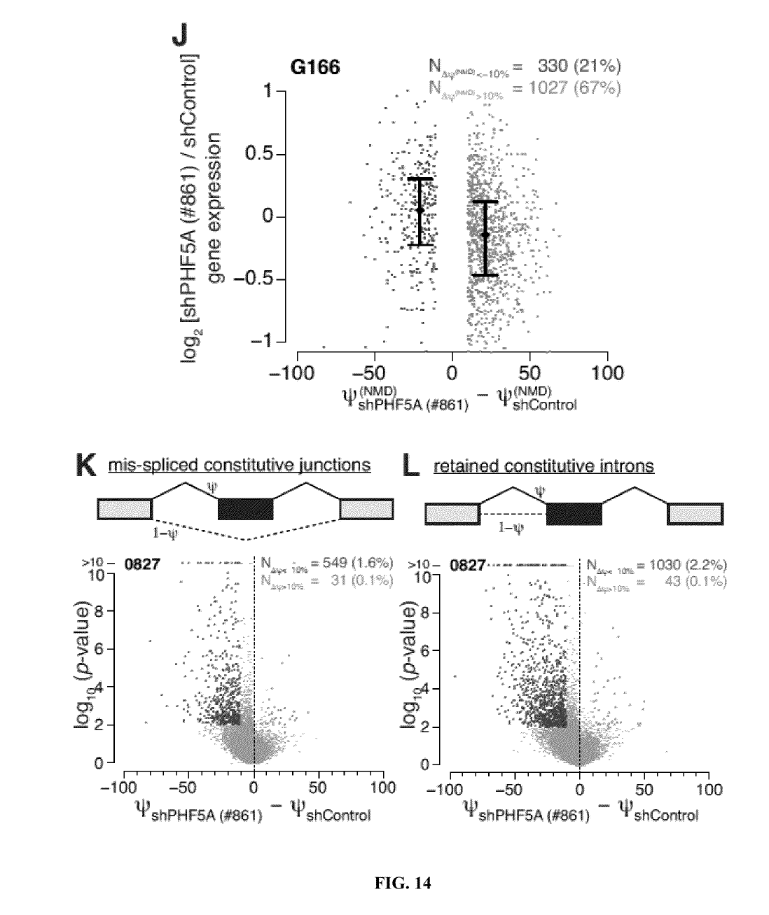

More specifically, severe RNA processing defects were found in many genes important for cell cycle progression, including CDC16, CDC20, CDC25C, CDC37, CDC45, and RCC2, in GSCs (G166 and 0827 cells) but not NSCs (CB660). For example, following PHF5α knockdown, the 3′-most constitutive exons of CDC20 were frequently skipped in G166 cells (FIG. 14A), and many constitutive exons in RCC2 were skipped in 0827 cells. If translated, these aberrant mRNAs would produce C-terminal truncated proteins that are unlikely to function normally in cell cycle progression. This severe dysregulation of cell cycle genes in GSCs, but not NSCs, indicates that aberrant splicing in GSCs following PHF5α knockdown may give rise to the observed mitotic arrest and inviability.

Further quantification of changes in isoform ratios was performed to determine whether splicing was globally dysregulated following PHF5α knockdown in NSCs and GSCs using only reads crossing splice sites, which is an approach that treats all splicing events with equivalent statistical power (Bradley et al., PLoS Biol. 10:e1001229, 2012). Classifying alternative splicing events as competing 5′ and 3′ splice sites, mutually exclusive exons, and cassette exons, PHF5α knockdown showed a global trend towards skipping of cassette exons—but few other splicing changes—in GSCs, but not NSCs (FIG. 14B-I). Most of the resulting splicing changes introduced in-frame stop codons into the mRNAs, strongly indicating that the splicing changes are aberrant, rather than functionally relevant, splicing. Genes expressing higher levels of isoforms with in-frame stop codons caused by PHF5α knockdown had lower average expression (FIG. 14J), consistent with degradation of the aberrant mRNAs by, for example, a nonsense-mediated decay (NMD) pathway.

Based on these findings, PHF5α may primarily function to facilitate exon recognition instead of regulating alternative splicing. Analyzing all annotated constitutive splice junctions in the human genome showed that PHF5α knockdown caused a broad shift toward alternative splicing of constitutive junctions (FIG. 14K) and retention of constitutive introns (FIG. 14L). Importantly, most such alternative splicing of constitutive junctions and almost all retention of constitutive introns will introduce in-frame stop codons, again resulting in non-functional mRNAs encoding truncated proteins.

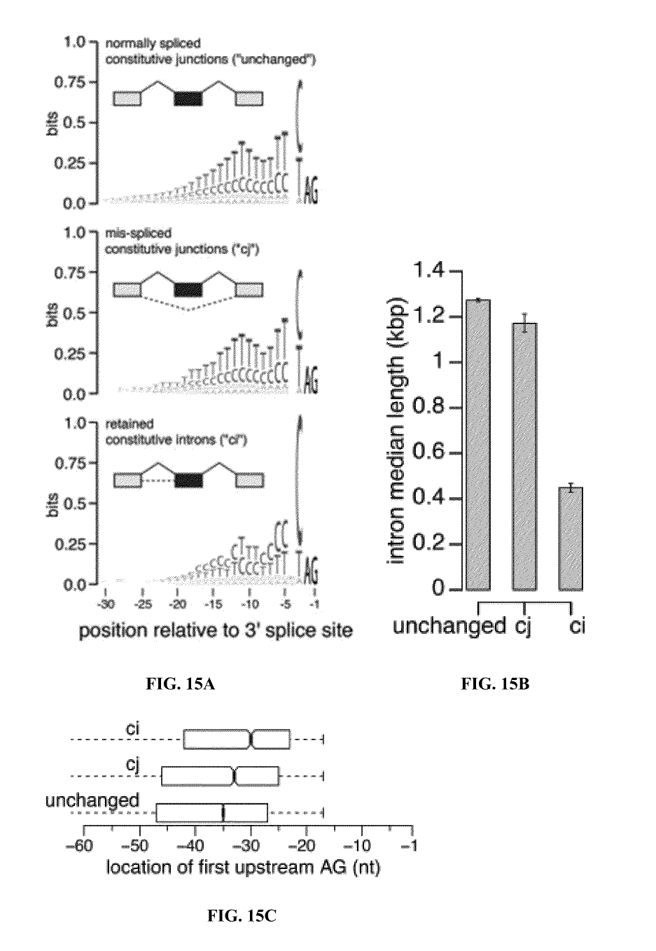

Only a relatively small subset of splice junctions was affected by PHF5α depletion in GSCs, indicating that the requirement for PHF5α is not universal across exons. The specific features characteristic of 3′ splice sites susceptible to abnormal splicing of constitutive junctions due to PHF5α knockdown in GSCs include slightly shorter, but otherwise normal, polypyrimidine tracts relative to unaffected 3′-splice sites; in contrast, 3′-splice sites associated with retained constitutive introns had unusual, C-rich tracts (FIG. 15A). Retained constitutive introns were short (FIG. 15B) and had unusually proximal branch points (FIG. 15C). While PHF5α is known as a core component of the spliceosome, it appears to be most important for recognition of an unusual class of exons with distinctive 3′-splice sites. Although these are hallmarks of many splice sites affected by PHF5α knockdown, other splice sites can also be affected.

To further examine whether PHF5α knockdown triggers cell cycle arrest by causing DNA damage, levels of pH2AX levels or phosphorylation of CHK1 and CHK2 were examined. Upon PHF5α knockdown in GSCs, no increase in pH2AX levels or phosphorylation of the DNA damage signaling proteins CHK1 and CHK2 was observed. These results indicate that the shPHF5α phenotype does not simply arise from a DNA damage response. Furthermore, BUB1B is not phosphorylated following PHF5α knockdown in GSCs, indicating that the spindle assembly checkpoint is also not being triggered.

Taken together, these data indicate that PHF5α is important for proper recognition of a specific, relatively small class of exons in GSCs. Knockdown of PHF5A causes defective RNA processing of thousands of essential genes, a subset of which is required for mitotic progression. Therefore, dysregulated RNA processing following knockdown of PHF5α results in GSC, but not normal NSC, non-viability.

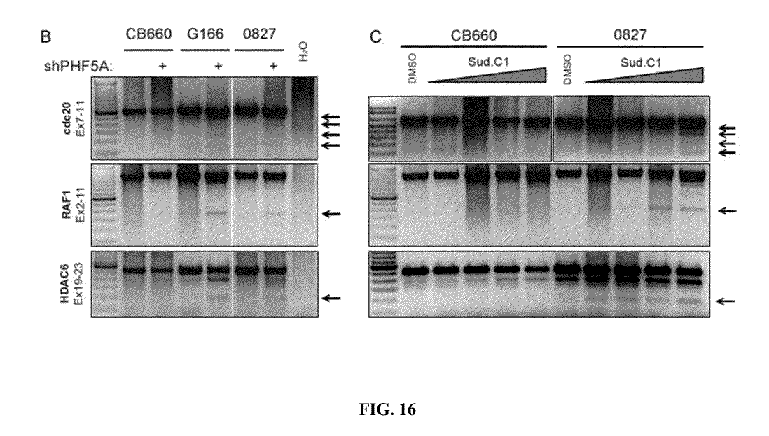

This example also describes the effects of two candidate small molecule inhibitors of the U2 snRNP complex, Spliceostatin A (SSA) and Sudemycin C1 (SudC1). SSA binds to and inhibits the U2 snRNP subunit SF3b, which contains PHF5A, resulting in a reduction in the fidelity of branch point recognition and a downregulation of genes important for cell division. (Kaida et al. 2007; Corrionero et al. 2011). SudC1 shares the consensus pharmacophore of SSA and pladienolide (Kotake et al. 2007) and also modulates RNA splicing (Lagisetti et al. 2008; Lagisetti et al. 2009; Fan et al. 2011). It was reasoned that if the most relevant GSC-specific function of PHF5A is its function in the splicing activity of the U2 snRNP, then these drugs should show a similar pattern of effects on RNA splicing in GSCs and NSCs. This was indeed the case. Treatment of GSCs with SudC1 resulted in dose-dependent GSC-specific splicing defects (FIG. 16). In addition, after PHF5A knockdown, multiple constitutive exons of the well characterized RTK/Ras signaling effector RAF1 and the cancer-associated deacetylase HDAC6 were skipped GSCs but not in normal NSCs.

Taken together, these results indicate that PHF5A is important for proper recognition of a specific, relatively small class of exons in GSCs. KD of PHF5A causes defective RNA processing of thousands of genes, a subset of which are essential for cell cycle progression. Given the broad splicing dysregulation that we observed, there are likely to be numerous cellular defects induced by PHF5A KD that contribute to the observed GSC inviability. This model is consistent with our observation that multiple methods of inhibiting U2 snRNP activity—including KD of other spliceosomal genes (below), as well as SudC 1 treatment—mimic the effects of PHF5A KD, even though these distinct perturbations are unlikely to lead to identical defects in RNA processing.

It was also discovered that depletion of PHF5A does not trigger the DNA damage response, but does cause splicing defects in GSCs. NSC or GSC cultures were infected with shCtrl or shPHF5A virus then selected with puromycin. Positive control cultures were treated with Cisplatin or Nocodozole for 24 hours. Equal cell lysates were run as western blots and incubated with the indicated phospho-specific antibodies. KD of PHF5A results in few changes in usage of competing 5′ splice sites, competing 3′ splice sites, or mutually exclusive exons in either NSCs or GSCs. However, PHF5A KD causes a broad increase in skipping of cassette exons in GSCs, but not NSCs.

### Example 8

### Screening of a Library of Candidate Agents

This example describes one suitable procedure to test for the ability of candidate agents to affect (e.g., inhibit) activity and/or expression of PHF5A, U2AF1 or DDX1, thereby allowing for identification of PHF5A, U2AF1 or DDX1 antagonists. Multiple splicing reporter assays published in the literature which could be adapted for this screen. See, e.g., Younis et al., Mol. Cell. Biol. 30(7): 1718-1728 (2010); Orengo et al., Nucleic Acids Research 34(22) e148 (2006); Nasim and Eperon, Nature Protocols, 1(2): 1022 (2006); Gurskaya et al., Analysis of Alternative Splicing of cassette exons at single-cell level using two fluorescent proteins; Nucleic Acids Res., 1-6 (2012); Xiao et al., Nat. Struct. & Mol. Biol., 16(10):1094-1101 (2009). A de-novo splicing reporter could also be generated either using a system like pSpliceExpress (Kishore et. al. Rapid generation of splicing reporters with pSpliceExpress. Gene. Volume 427, Issues 1-2, 31 Dec. 2008, Pages 104-110) using traditional molecular biology and cloning techniques. Reporter constructs may utilize one or more detectable reporter genes such as, but not limited to, luciferase of a fluorescent protein. The splicing reporter will produce a primary transcript that includes exonic sequences separated by intronic sequence(s) that may be removed from the transcript by cellular splicing machinery. The splicing of the reporter transcript, and consequent inclusion or exclusion of the intervening sequence, can modulate the detectability of the reporter gene, either by completing or disrupting the final protein function. The sequence of the 3′ splice sites would be chosen to best facilitate the screens below.

The screen could be set up to detect at least one of two novel hallmarks of PHF5A inhibition: a) greater splicing perturbation in cancer cells compared to normal cells, or b) reduced recognition of introns with C-rich 3′ splice sites. Splicing perturbation in cancer compared to normal cells would be one method. Normal and cancer cell lines expressing the above reporter constructs would be seeded in multi-well plates and exposed to test compounds for a limited amount of time. Reporter activity would be measured and compared to controls for each cell line, as well as across cell lines to identify compounds that selectively reduce splicing of the reporter gene in the cancer cells compared to the non-cancerous cells. C-rich 3′ splice site recognition would be another method. Reporter constructs and reporter cell lines would be generated that differ in the sequence of the 3′ splice sites within the reporter gene(s). Cells would be screened as above to identify compounds that impede proper splicing of reporter genes containing C-rich splice sites but not canonical splice sites.

Binding assays would be further conducted to identify candidate agents that can affect (e.g., inhibit) activity and/or expression of PHF5A, U2AF1 or DDX1. For example, purified PHF5A protein can be generated by expression of either a) intact PHF5A protein, b) PHF5A fused to another protein, or c) affinity tagged PHF5A (which could be tagged using any of multiple known epitope such as FLAG, HA, His, or a biotinylation sequence. PHF5A protein could be produced using in vitro transcription systems (many commercially available also) or using a cell expression systems. Alternatively endogenous PHF5A could be isolated from cell lysates using immunopurification with, e.g., commercially available PHF5A antibodies, followed by isolation of PHF5A based on physical or biochemical properties (ie. Size exclusion columns). The purified PHF5A protein (or U2AF1 or DDX1) may be then tested for candidate compound binding using techniques such as Surface Plasmon Resonance or radioligand binding, as well as other binding assays generally well known in the art.

In one example, candidate agents (e.g., lead compounds) active as inhibitors of PHF5α can interact with the PHF5α through a chemical interaction that can be characterized by a number of methods broadly known to those skilled in the art of drug discovery and characterization. One such method that is commonly employed to characterize drug-target interactions is surface plasmon resonance (SPR). SPR biosensors can be used to characterize the interaction of protein, peptide, or small molecule drug candidates with their target protein in real-time, without the need for fluorescent or radioisotopic labeling of the drug. See, e.g., Myszka D G and Rich R L, Pharmaceutical Sci & Tech Today 3(9):310-317 (2000). In a typical experiment, the drug target is immobilized on the biosensor surface, for example by direct amine coupling, and the drug lead candidates are passed over the surface through a microfluidic flow cell. Interaction of drug with drug target is assessed by monitoring changes in the refractive index of the solvent layer near the surface of the biosensor triggered by association of the drug with the target. The surface plasmon resonance methods can be used to measure the affinity of the interaction and the kinetics of association and dissociation of drug with target. Other assay systems can also be modified and used for screening candidate agents described herein. See, e.g., Markgren P O, Hamalainen M, and Danielson U H. Anal Biochem 265(2):340-350 (1998).

With PHF5α, for example, purified protein can be immobilized on the SPR biosensor surface, and protein, peptide, or small molecule leads that bind to the immobilized PHF5α are detected through by measuring the refractive index of the solvent layer, thus providing an analytical method capable of confirming that the selected drug candidates interact specifically with the target (PHF5α), and providing data to rank compounds based on their affinity and association and dissociation rates. A similar procedure would be used for U2AF1 or DDX1.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treating a cellular hyperproliferative disorder associated with an oncogenic pathway, the method comprising:
a) identifying at least one candidate agent that is a PHF5α antagonist, a U2AF1 antagonist, a DDX1 antagonist, or a combination thereof;
b) determining whether a subject is suffering from a cellular hyperproliferative disorder associated with the oncogenic pathway; and
c) if the subject is suffering from a cellular hyperproliferative disorder associated with the oncogenic pathway, administering to the subject in need thereof a therapeutically effective amount of the PHF5α antagonist, the U2AF1 antagonist, the DDX1 antagonist, or the combination thereof.

2-3. (canceled)

4. The method of claim 1, wherein the oncogenic pathway comprises an aberrant Ras pathway.

5. (canceled)

6. The method of claim 1, wherein the subject has a tumor, and wherein the determining comprises determining whether the tumor comprises a mutation or dysregulation in Ras, RTK, Raf, MAPK, ERK, MEK, MKK, AKT, PI3K, Myc, or any combination thereof.

7. The method of claim 1, wherein the cellular hyperproliferative disorder is a glioma, glioblastoma multiforme, sarcoma, liver cancer, pancreatic ductal adenocarcinoma, adenocarcinoma, colorectal cancer, cervical cancer, or prostate cancer.

8-10. (canceled)

11. The method of claim 1, wherein the subject is a human.

12. The method of claim 1, wherein the at least one candidate agent comprises a polypeptide, a polynucleotide, or a small molecule compound.

13. The method of claim 1, wherein the identifying comprises:
a) providing at least one candidate agent;
b) contacting the at least one candidate agent with proliferating cancer cells having an oncogenic pathway;
c) determining whether cell cycle arrest, dysregulated cell cycle progression, dysregulated RNA processing, or a combination thereof is generated in proliferating cancer cells due to inhibition of PHF5α, U2AF1, DDX1, or a combination thereof, wherein cell cycle arrest, dysregulated cell cycle progression, dysregulated RNA processing, or a combination thereof indicates inhibition of PHF5α, U2AF1, DDX1, or a combination thereof by the at least one candidate agent; and
d) determining whether the at least one candidate agent binds to PHF5α, U2AF1, DDX1, or a combination thereof, thereby identifying the PHF5α antagonist, U2AF1 antagonist, DDX1 antagonist, or the combination thereof.

14-27. (canceled)

28. The method of claim 1, wherein the PHF5α antagonist, U2AF1 antagonist, DDX1 antagonist, or a combination thereof is used in combination with a chemotherapeutic agent or a spliceosome inhibitor.

29. The method of claim 28, wherein the spliceosome inhibitor is sudemycin, spliceostatin, FR901464, pladienolide, E7107, herboxidine, meayamycin, or a derivative or analog thereof.

30-43. (canceled)

44. A method for identifying a PHF5α antagonist, a U2AF1 antagonist, a DDX1 antagonist, the method comprising:
a) providing at least one candidate agent;
b) contacting the at least one candidate agent with proliferating cancer cells associated with an oncogenic pathway;
c) determining whether the at least one candidate agent inhibits proliferation of the cancer cells, wherein inhibition indicates inhibition of PHF5α, U2AF1, DDX1, or a combination thereof, by the at least one candidate agent; and
d) determining whether the at least one candidate agent binds to PHF5α, U2AF1, DDX1, or a combination thereof, thereby identifying the PHF5α antagonist, U2AF1 antagonist, DDX1 antagonist, or the combination thereof.

45. A method of treating a subject having a cellular hyperproliferative disorder associated with an oncogenic pathway, the method comprising administering a pharmaceutical composition comprising a therapeutically effective amount of the PHF5α antagonist, U2AF1 antagonist, DDX1 antagonist, or the combination thereof identified in claim 44.

46-49. (canceled)

50. The method of claim 44, wherein the PHF5α antagonist, the U2AF1 antagonist, the DDX1 antagonist, or the combination thereof directly inhibits activity and/or expression of PHF5α, U2AF1, DDX1, or a combination thereof.

51-74. (canceled)

75. A method for identifying a SF3b antagonist, the method comprising:
a) providing at least one candidate agent;
b) contacting the at least one candidate agent with proliferating cancer cells;
c) determining whether cell cycle arrest, dysregulated cell cycle progression, dysregulated RNA processing, or a combination thereof is generated in proliferating cancer cells due to inhibition of SF3b, wherein cell cycle arrest, dysregulated cell cycle progression, dysregulated RNA processing, or a combination thereof indicates inhibition of SF3b by the at least one candidate agent; and
d) determining whether the at least one candidate agent binds to SF3b, thereby identifying the SF3b antagonist.

76. A method of treating a subject having a cellular hyperproliferative disorder associated with an oncogenic pathway, the method comprising administering a pharmaceutical composition comprising a therapeutically effective amount of the SF3b antagonist identified in claim 75.

77-82. (canceled)

83. The method of claim 75, wherein the dysregulated RNA processing comprises global exon skipping, aberrant constitutive junction splicing, constitutive intron retention, or a combination thereof.

84-85. (canceled)

86. The method of claim 45, wherein the cellular hyperproliferative disorder is a glioma, glioblastoma multiforme, sarcoma, liver cancer, pancreatic ductal adenocarcinoma, adenocarcinoma, colorectal cancer, cervical cancer, or prostate cancer.

87. The method of claim 45, wherein the subject is a human.

88. The method of claim 76, wherein the cellular hyperproliferative disorder is a glioma, glioblastoma multiforme, sarcoma, liver cancer, pancreatic ductal adenocarcinoma, adenocarcinoma, colorectal cancer, cervical cancer, or prostate cancer.

89. The method of claim 76, wherein the subject is a human.
